(19) United States
(12) Patent Application Publication
(10) Pub. No.: US 2010/0179567 A1
(43) Pub. Date: Jul. 15, 2010

(54) CLOSURE DEVICES, SYSTEMS, AND METHODS

(75) Inventors: Laveille K. Voss, Belmont, CA (US); Sung-Woo Min, Fremont, CA (US); Alexander S. Wong, Redwood City, CA (US); Douglas H. Mehl, Redwood City, CA (US); David J. Milazzo, Santa, CA (US)

Correspondence Address: WORKMAN NYDEGGER, 1000 EAGLE GATE TOWER,, 60 EAST SOUTH TEMPLE, SALT LAKE CITY, UT, 84111, US

(73) Assignee: ABBOTT VASCULAR INC., Santa Clara, CA (US)

(21) Appl. No.: 12/684,542

(22) Filed: Jan. 8, 2010

Related U.S. Application Data
(60) Provisional application No. 61/143,751, filed on Jan. 9, 2009.

(51) Int. Cl. A61B 17/10; A61B 17/08

(52) U.S. Cl. 606/139; 606/151

(57) ABSTRACT

The present disclosure includes anchor members configured to locate and/or anchor tissue surrounding a body lumen opening comprising. In one implementation, the anchor member can include an elongate portion configured to be manipulated by a user and an anchor portion. In a further implementation, the anchor portion can have one or more contracted configurations capable of passing through a body lumen opening and one or more expanded configurations capable of anchoring tissue surrounding a body lumen opening.

### CROSS-REFERENCE TO RELATED APPLICATIONS

This patent application claims the benefit of and priority to U.S. Provisional Patent Application Ser. No. 61/143,751, entitled “Vessel Closure Devices and Methods,” filed Jan. 9, 2009, the disclosure of which is incorporated herein by reference in its entirety.

### BACKGROUND

1. Technical Field

The present disclosure relates generally to medical devices and their methods of use. In particular, the present disclosure relates to vessel closure systems and devices and corresponding methods of use.

2. The Technology

Catheterization and interventional procedures, such as angioplasty or stenting, generally are performed by inserting a hollow needle through a patient's skin and tissue into the vascular system. A guidewire may be advanced through the needle and into the patient's blood vessel accessed by the needle. The needle is then removed, enabling an introducer sheath to be advanced over the guidewire into the vessel, e.g., in conjunction with or subsequent to a dilator.

A catheter or other device may then be advanced through a lumen of the introducer sheath and over the guidewire into a position for performing a medical procedure. Thus, the introducer sheath may facilitate introducing various devices into the vessel, while minimizing trauma to the vessel wall and/or minimizing blood loss during a procedure.

Upon completing the procedure, the devices and introducer sheath would be removed, leaving a puncture site in the vessel wall. Traditionally, external pressure would be applied to the puncture site until clotting and wound sealing occur; however, the patient must remain bedridden for a substantial period after clotting to ensure closure of the wound. This procedure may also be time consuming and expensive, requiring as much as an hour of a physician's or nurse's time. It is also uncomfortable for the patient and requires that the patient remain immobilized in the operating room, catheter lab, or holding area. In addition, a risk of hematoma exists from bleeding before hemostasis occurs. Although some closure systems may be available, they provide limited control to flexibility to the operator, which may lead to improper or undesirable closure of the puncture site.

### BRIEF SUMMARY

The present disclosure includes anchor members configured to locate and/or anchor tissue surrounding a body lumen opening comprising. In one implementation, the anchor member can include an elongate portion configured to be manipulated by a user and an anchor portion. In a further implementation, the anchor portion can have one or more contracted configurations capable of passing through a body lumen opening and one or more expanded configurations capable of anchoring tissue surrounding a body lumen opening.

The anchor member can also include one or more shape memory materials. In a further implementation, the anchor member can include a shape memory wire, wherein the anchor portion includes a portion of the shape memory wire being heat set into an aneurysm coil or bird's nest having a plurality of non-uniformly shaped and oriented loops of the shape memory wire. In a yet further implementation, the anchor member can include a shape memory wire, wherein the anchor portion includes a portion of the shape memory wire being heat set into a spiraling configuration having a plurality of spirals extending radially outwardly from a longitudinal axis of the elongate portion. In a yet further implementation, the anchor portion can include an expandable mesh structure.

In one implementation, the anchor portion of the anchor member can include a plurality of hinged struts configured to expand radially outwardly, and wherein the elongate portion includes a first elongate member disposed through a generally tubular second elongate member. In a yet further implementation, the first elongate member is connected to a distal end of the anchor portion and the second elongate member is connected to a proximal end of the anchor portion and selective relative movement between the first elongate member and second elongate member causes the anchor portion to selectively expand and contract.

In one implementation, the anchor member can include a flexible membrane disposed around at least a portion of the anchor portion. In a further implementation, the anchor portion can include an expandable coil of shape memory wire. In a yet further implementation, the anchor member can be a generally tubular member having a straight portion forming the elongate portion and a looped portion forming the anchor portion configured to pass through an opening by rotation of the anchor member. In an additional implementation, the anchor member can be initially disposed in a delivery lumen in a contracted configuration and can be configured to superelastically deploy through a lateral opening in the delivery lumen into an expanded configuration.

In one implementation, the elongate portion of the anchor member can include a mandrel having one or more teeth disposed along a length thereof and the anchor portion can include one or more rotatable projections each having one or more recesses with sizes and shapes corresponding to the sizes and shapes of the one or more teeth of the elongate portion. In a further implementation, the elongate portion can be configured to rotate the one or more projections of the anchor portion by moving longitudinally relative to the anchor portion with the teeth of the elongate portion engaging the recesses of the one or more projections. In a yet further implementation, the anchor member can include a delivery lumen and one or more hinges connected to the delivery lumen and passing through the one or more projections to facilitate rotation of the projections about the hinges.

In one implementation, the anchor portion can include a wire mesh basket having an open proximal end and a closed distal end. In a further implementation, the distal end of the elongate portion can be connected to the distal end of the anchor portion.

In one implementation, the elongate portion can include a first elongate member and a generally tubular second elongate member, wherein the first elongate member is disposed at least partially through the second elongate member. In a further implementation, the anchor portion can include one or more projections configured to be movable between a contracted configuration and an expanded configuration. In a yet further implementation, the distal end of the first elongate member can be connected to a distal end of the anchor portion and a distal end of the second elongate member can be connected to a proximal end of the anchor portion. In an additional implementation, a distal end of the first elongate member can be connected to a center or proximal end of the anchor portion, wherein the anchor portion has a free outside or distal end. In a further implementation, the one or more projections can have a ribbon- or strip-like configuration with one end thereof connected to the first elongate member and an opposite end thereof connected to the second elongate member. In a yet further implementation, the anchor portion has a first contracted configuration wherein the anchor portion is retracted into the second elongate member and a second contracted configuration wherein the anchor portion is elongated in a distal direction. In an additional implementation, the first elongate member can include a collar positioned thereon and the second elongate member further includes one or more stops disposed on the inner surface thereof, and wherein the collar and stops are configured to limit relative longitudinal movement between the first elongate member and the second elongate member. In a further implementation, the collar and stops can be configured to allow sufficient distal movement of the first elongate member to deploy the anchor portion and sufficient proximal movement of the first elongate member to retract the anchor portion into the second elongate member. In a yet further implementation, the second elongate member can be a guidewire.

Implementations of the present disclosure can also include a closure system. In one implementation, the closure system can include a handle member, a tube set configured to deliver and/or deploy a closure element, a plunger member movably coupled to the handle member, and an anchor member disposed at least partially within the tube set.

These and other advantages and features of the present disclosure will become more fully apparent from the following description and appended claims, or may be learned by the practice of the disclosure as set forth hereinafter.

### BRIEF DESCRIPTION OF THE DRAWINGS

To further clarify at least some of the advantages and features of the present disclosure, a more particular description of the disclosure will be rendered by reference to specific embodiments thereof which are illustrated in the appended drawings. It is appreciated that these drawings depict only illustrated embodiments of the disclosure and are therefore not to be considered limiting of its scope. The disclosure will be described and explained with additional specificity and detail through the use of the accompanying drawings in which:

FIG. 1 discloses a closure system in accordance with one example embodiment.

FIGS. 2A-2D disclose example steps of operating the closure system of FIG. 1 in accordance with a further embodiment.

FIGS. 3A-3B disclose an example anchor member in accordance with a yet further embodiment.

FIGS. 4A-4B disclose an example anchor member in accordance with an additional example embodiment.

FIGS. 5A-5D disclose example steps of operating the anchor member of FIGS. 3A-3B in accordance with one embodiment.

FIGS. 6A-6G disclose example steps of closing a body lumen opening in accordance with a further embodiment.

FIG. 7 discloses an example anchor member in accordance with one embodiment.

FIG. 8 discloses an example anchor member in accordance with a further embodiment.

FIGS. 9A-9C disclose example steps of deploying an example anchor member in accordance with a yet further embodiment.

FIG. 10 discloses an example anchor member in accordance with an additional embodiment.

FIG. 11 discloses an example anchor member in accordance with one embodiment.

FIGS. 12A-12B disclose an example anchor member in accordance with a further embodiment.

FIGS. 13A-13B disclose an example anchor member in accordance with a yet further embodiment.

FIGS. 14A-14B disclose an example anchor member in accordance with an additional embodiment.

FIGS. 15A-15B disclose an example anchor member in accordance with one embodiment.

FIG. 16 discloses an example anchor member in accordance with a further embodiment.

FIG. 17 discloses an example anchor member in accordance with a yet further embodiment.

FIG. 18A discloses the example anchor member of FIG. 17 in a deployed/expanded configuration.

FIG. 18B discloses the example anchor member of FIG. 17 in a collapsed configuration in accordance with one embodiment.

FIG. 18C discloses the example anchor member of FIG. 17 in a collapsed configuration in accordance with an additional embodiment.

FIGS. 19A-19C disclose an example anchor member in accordance with a yet further embodiment.

FIGS. 20A-20C disclose an example anchor member in accordance with an additional embodiment.

FIGS. 21A-21B disclose an example anchor member in accordance with a further embodiment.

It should be noted that the figures are not drawn to scale and that elements of similar structures or functions are generally represented by like reference numerals for illustrative purposes throughout the figures. It also should be noted that the figures are only intended to facilitate the description of example configurations of the present disclosure.

### DETAILED DESCRIPTION

The present disclosure relates to devices, systems, and methods for closing an opening in a body lumen. In one example embodiment, a closure system of the present disclosure may allow an operator to quickly and efficiently close a body lumen opening while simultaneously providing the operator with a greater measure of control and flexibility in positioning and anchoring the closure system than previously available. For example, the closure system may allow an operator to achieve a more intimate securement of a closure element in the tissue surrounding a body lumen opening. In a yet further embodiment, the closure system may be compatible with a wider range of body lumen wall thicknesses, thereby taking into account the possibility of calcifications or scar tissue in the lumen wall. In addition, the closure system may be configured to advance into a body lumen opening over a guidewire. Furthermore, the closure system may be compatible with a variety of sizes of body lumen openings and tissue tracts.

Embodiments of the disclosure further relate to a device closure system with a removable anchor. In one example, the anchor can be deployed from a contracted state to an expanded state. When in the expanded state, the anchor can be used to locate an opening in a vessel (e.g., an arteriotomy) when deploying, for example, a closure element, such as a clip or staple. The anchor, in conjunction with a tube set in the closure system, may sandwich the tissue surrounding the opening in the vessel. This effectively locates the opening and aids in effective and proper deployment of the closure element.

The closure system may then retract or remove the anchor during use of the closure system, leaving the arteriotomy or opening at least substantially closed or sealed by the closure element. During removal, the anchor can deform without dislodging the closure element. More specifically in one embodiment, the anchor is withdrawn back into the tube set and into the pre-deployed state. Thus, the closure system and close an opening in a body lumen using a removable anchor.

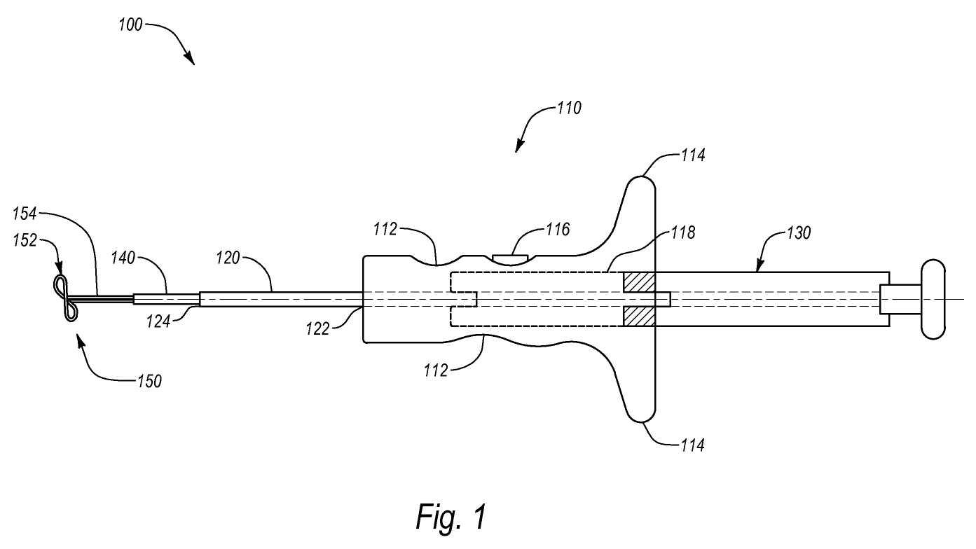

Reference is now made to FIG. 1 which illustrates a closure system 100 in accordance with an implementation of the present disclosure. The closure system 100 may be configured to close an opening in a body lumen. The closure system 100 may include a handle member 110, a tube set 120 coupled to the handle member 110, a plunger member 130, an inner lumen 140, and an anchor member 150 disposed at least partially within the inner lumen 140. An operator, such as a physician, may utilize the closure system 100 and the elements thereof to close an opening in a body lumen. For example, as will be explained in more detail below, the plunger member 130 may be used to deploy the anchor member 150 to locate the distal surface of a lumen wall and position the closure system 100 relative to a body lumen opening. Thereafter, the handle member 110 and tube set 120 may be used to deliver a closure element, such as a clip or staple, and deploy the closure element into the tissue of the body lumen wall to close or substantially close the body lumen opening.

The handle member 110 of the closure system 100 may be configured to assist an operator, such as a physician, to grip, manipulate, advance, and/or operate the closure system 100 in order to close a body lumen opening. In particular, the handle member 110 may have a shape and size that conforms to the shape and size of a human hand. The handle member 110 may also include a number of indentations 112 configured to at least partially receive the fingers and/or thumbs of the operator. The indentations 112 may assist the operator to grip and manipulate the handle member 110 and closure system 100. The handle member 110 may also include one or more flanges 114 to assist an operator to grip, advance, and/or retract the handle member 110 and/or closure system 100.

The handle member 110 may also include any number of mechanisms necessary to deploy a closure element. For example, the handle member 110 may include a button 116 operatively associated with one or more mechanisms configured to deploy a closure element. The button 116 may be positioned in or proximate to one of the one or more indentations 112. In a further embodiment, the button 116 may be operatively associated with one or more elements of the tube set 120 configured to deploy the closure element 100. As a result, an operator may depress the button 116 in order to push, fire, or eject a closure element from the tube set 120 into the tissue of a body lumen to close a body lumen opening.

In a further embodiment, the handle member 110 may include a recess 118 configured to receive at least a portion of the plunger member 130. The recess 118 may be further configured to allow the plunger member 130 to move in a longitudinal direction relative to the handle member 110. In particular, the recess 118 may allow the plunger member 130 to move both distally and proximally relative to the handle member 110. For example, the recess 118 may have a cross-sectional shape similar to, but slightly larger than, the cross sectional shape of the plunger member 130. As a result, the plunger member 130 may slide into and out of the recess 118 to move relative to the handle member 110.

The handle member 110 may include any number of rigid or semi-rigid materials. For example, the handle member 110 may include any number of polymers, plastics, metals, composites, other similar materials, or combinations thereof.

The tube set 120 may be coupled to and/or partially disposed within the handle member 110. The tube set 120 may have a proximal end 122 coupled to the handle member 110 and opposite a distal end 124. The tube set 120 may be configured to contain, deliver, and/or deploy a closure element. In particular, the tube set 120 may include one or more tubular members and/or other mechanisms configured to house, advance, push, fire, and/or eject the closure element. For example, the tube set 120 may include a pusher tube, a garage tube, a carrier tube, and/or other similar elements. In one embodiment, the tube set 120 may include a spring-loaded pusher member configured to deploy the closure element when released or activated.

The closure element may be disposed within the tube set 120 in an initial, open configuration and may be configured to be deployed from the tube set 120 and move to a deployed, closed configuration. In particular, in one embodiment, the closure element may store sufficient energy, while in its initial, open configuration, to engage the tissue of and close an opening in a lumen wall. For example, the closure element may include any of a number of shape memory and/or superelastic materials and may be set to elastically return to a deployed, closed configuration from any other configuration. In one embodiment, the closure element may include nitinol. In a further embodiment, the closure element may be a clip, staple, or other closure element.

The closure system 100 may also include an inner lumen 140. The inner lumen 140 may be disposed at least partially within the tube set 120, the handle member 110, and/or the plunger member 130. In a further implementation, the inner lumen 140 may be movable, such as slidable, with respect to the tube set 120, the handle member 110, and/or the plunger member 130. As a result, the inner lumen 140 may move either distally or proximally relative to the tube set 120, the handle member 110, and/or the plunger member 130.

The inner lumen 140 may be configured to house and deliver the anchor member 150 to or away from a body lumen opening. In a further embodiment, the inner lumen 140 may be integrated into or replaced by an element of the tube set 120. The inner lumen 140 may include any number of flexible or semi-rigid materials. For example, the inner lumen may include one or more polymers, elastomers, plastics, metals, composites, other similar materials, or combinations thereof.

As introduced above, the closure system 100 may include an anchor member 150. The anchor member 150 may be configured to locate, position the closure system 100 relative to, and/or anchor the tissue surrounding a body lumen opening. The anchor member 150 may include an anchor portion 152 and an elongate portion 154. The anchor portion 152 may be configured to be positioned and/or anchored against the distal surface of a lumen wall. The elongate portion 154 may be coupled to the anchor portion 152 and may be configured to control, deploy, position, stabilize, and/or retract the anchor portion 152. In particular, the elongate portion 154 may extend away from the anchor portion 152 in a proximal direction through the inner lumen 140, the tube set 120, the handle member 110, and/or the plunger member 130. In a further embodiment, the elongate portion 154 may be coupled at its proximal end 122 to the plunger member 130. In a yet further embodiment, the elongate portion 154 may be selectively detachable from and recouplable to the plunger member 130.

The anchor portion 152 of the anchor member 150 may be disposed in an initial, contracted configuration within the inner lumen 140. The elongate portion 154 of the anchor member 150 may extend proximally from the anchor portion 152 to the plunger member 130. In addition, the elongate portion 154 may transfer forces from the plunger member 130 to the anchor portion 152. Accordingly, by advancing the plunger member 130 or elongate portion 154 in a distal direction relative to the inner lumen 140 an operator may deploy the anchor portion 152 of the anchor member 150 from the distal end of the inner lumen 140. Retracting the plunger member 130 in a proximal direction may position and/or anchor the anchor portion 152 against a distal surface of a lumen wall. In a further embodiment, further retracting the plunger member 130 in a proximal direction may retract the anchor portion 152 of the anchor member 150 from the body lumen and/or into the inner lumen 140 or tube set 120.

The anchor portion 152 of the anchor member 150 may be configured to move from an initial, contracted configuration within the inner lumen 140 to a deployed, expanded configuration once deployed from the inner lumen 140. To facilitate movement from an initial, contracted configuration to a deployed, expanded configuration, the anchor portion 152 of the anchor member 150 may include one or more superelastic or shape memory materials such as shape memory alloys. For example, and as will be explained in more detail below, the anchor portion 152 be heat set in a deployed, expanded configuration. The anchor portion 152 may then be elastically deformed into an initial, contracted configuration contracted and disposed within the inner lumen 140. In its initial, contracted configuration, the anchor portion 152 may store sufficient energy to return to its deployed, expanded configuration once released from the inner lumen 140.

In one embodiment, a user may operate the plunger member 130 to deploy and/or retract the anchor member 150. For example, the plunger member 130 may be configured to at least partially receive the tube set 120 and/or the inner lumen 140. In a further embodiment, the plunger member 130 may also be configured to receive a portion of the anchor member 150 and/or a guidewire. In a further embodiment, the inner lumen 140 and/or anchor member 150 may be coated to minimize friction within the inner lumen 140 to ease deployment.

The proximal end 122 of the plunger member 130 may be configured to be gripped and/or operated by an operator such as a physician. For example, an operator may grip the handle member 110 with a first hand and grip the proximal end of the plunger member 130 with a second hand in order to advance or retract the plunger member 130 relative to the handle member 110. As a result, the operator may deploy the anchor portion 152 of the anchor member 150 from the inner lumen 140 and/or position the anchor portion 152 against a distal surface of a lumen wall thereby locating the body lumen opening to be closed.

Thereafter, the operator may advance the handle member 110 in a distal direction relative to the plunger member 130 and inner lumen 140 to position the distal end 124 of the tube set 120 against a proximal surface of the lumen wall. By so doing, the operator may facilitate the closure of the body lumen opening by at least partially gripping, sandwiching, and/or immobilizing the tissue surrounding the body lumen opening. The operator may then deploy a closure element into the tissue of the lumen wall to close the body lumen opening.

The shape of the plunger member 130 may correspond with the shape of the recess 118 to facilitate relative movement between the handle member 110 and the plunger member 130. For example, the cross sectional shape of both the plunger member 130 and the recess 118 may be any shape desired such as circular, triangular, rectangular, or other shapes, or combinations thereof. In addition, the length of the plunger member 130 and the corresponding depth of the recess 118 may be any length and depth desired to allow sufficient relative movement between the plunger member 130 and handle member 110. For example, the length of the plunger member 130 and the corresponding depth of the recess 118 may be sufficient to allow deployment of the anchor portion 152 from the inner lumen 140.

In a further embodiment, the closure system 100 may include a self-tensioning mechanism configured to automatically provide tension in the anchor member 150 once the anchor portion 152 has deployed. For example, in one embodiment, the handle member 110 may include a spring mechanism disposed in the recess 118 and configured to resist and/or counteract movement of the plunger member 130 in a distal direction relative to the handle member 110. In particular, advancing the plunger member 130 in a distal direction relative to the handle member 110 may transfer energy to the spring mechanism, which may be released once the operator releases the plunger member 130.

As a result, the spring mechanism may move the plunger member 130 in a proximal direction relative to the handle member 110 thereby retracting the anchor portion 152 in a proximal direction, thereby automatically engaging the distal surface of a lumen wall, and/or advancing the handle member 110 and tube set 120 in a distal direction, thereby engaging the proximal surface of the lumen wall. The spring mechanism can also create sufficient tension within the anchor member 150 to produce a desired pressure on the tissue of the lumen wall between the anchor portion 152 and the tube set 120. Accordingly, the closure system 100 may automatically and efficiently create the desired sandwiching or immobilizing force on the tissue surrounding the body lumen opening. In addition, the spring mechanism may make it unnecessary for the operator to provide the movement or force necessary to position the closure system 100 relative to the body lumen opening. In additional embodiments, any other self-tensioning mechanism may be included in the closure system 100 to produce to desired tension in the anchor member 150 and force upon the tissue surrounding the body lumen opening. In a yet further embodiment, the plunger member 130 and closure system 100 may have a click or ratchet function similar to that of a “click” pen.

In a yet further embodiment, the closure system 100, or the elements thereof, may include a mechanism for determining the thickness of a lumen wall and/or the distance between the anchor portion 152 and the distal end 124 of the tube set 120. For example, the plunger member 130 may have a plurality of indicator lines along the length thereof. The indicator lines may be positioned and marked to indicate the position of the deployed anchor portion 152 relative to the distal end 124 of the tube set 120. In particular, the number of indicator lines exposed as the plunger member 130 is retracted may indicate the thickness of the tissue surrounding the body lumen opening being closed. The indicator lines may be calibrated so that they read zero thickness when the anchor portion 152 is position directly against the distal end 124 of the tube set 120. As a result, the operator may refer to the indicator lines to determine the position of the anchor portion 152 relative to the distal end 124 of the tube set 120 and/or the thickness of the tissue surrounding a body lumen opening.

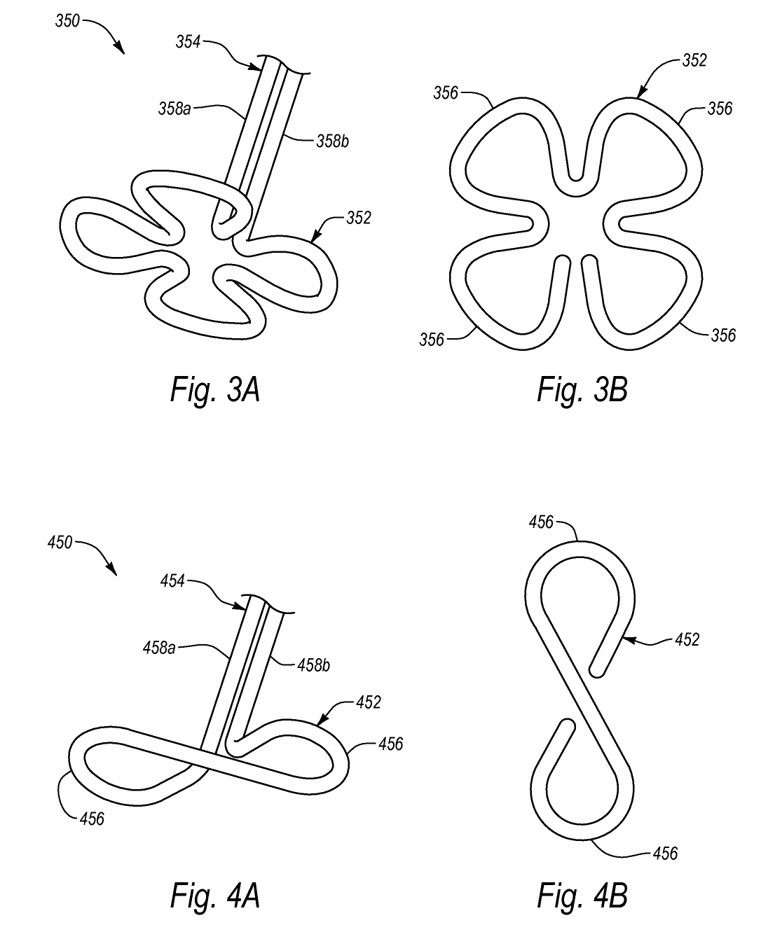

Additionally, the anchor member 150 may incorporate at least one component of the anchor members 350, 450, 550, 650, 750, 850, 950, 1050, 1150, 1250, 1350, 1450, 1550, 1650, 1750, 1950, 2050, and 2150 described in connection with FIGS. 3-21, respectively.

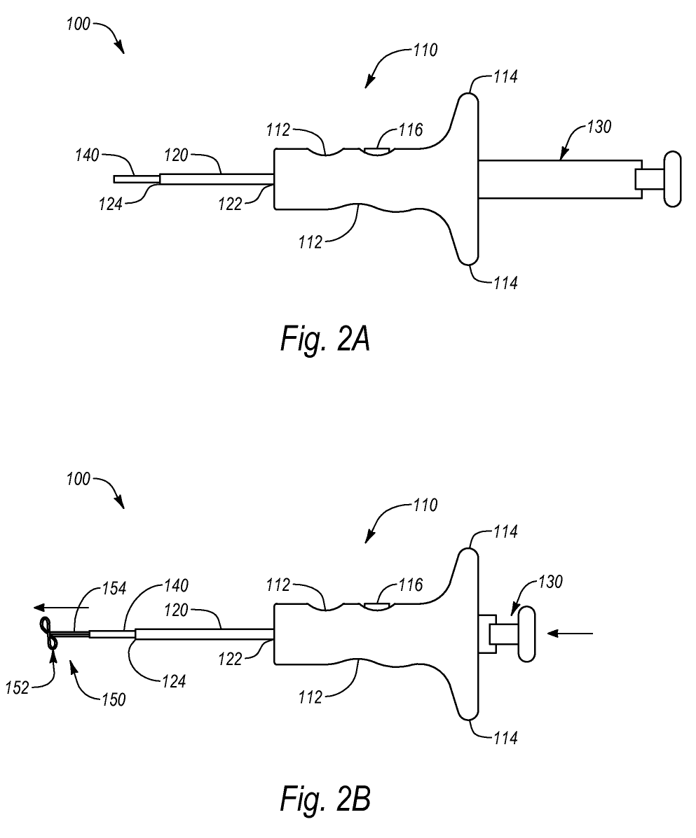

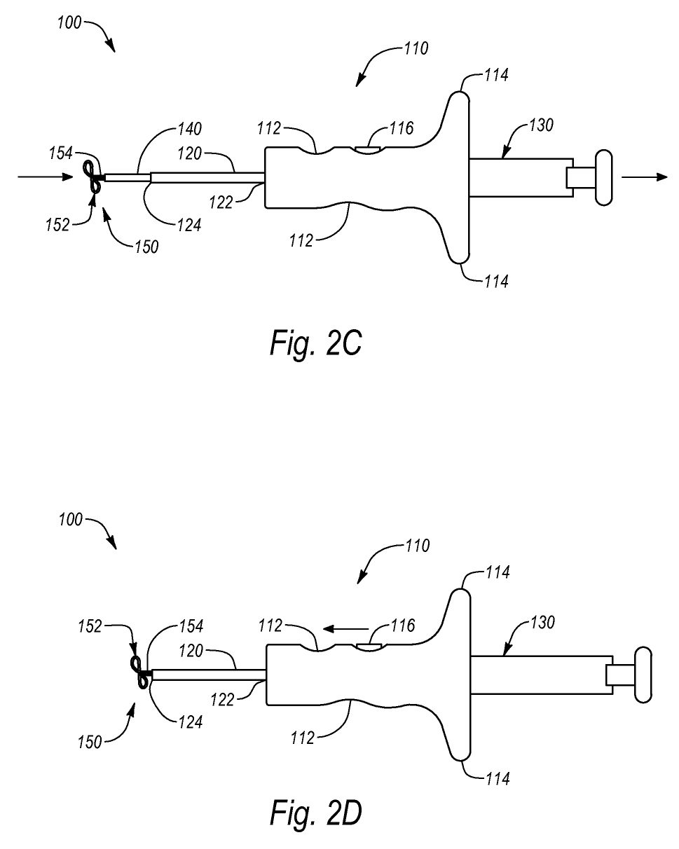

Reference is now made to FIGS. 2A-2D, which illustrate an example method of operating the closure system 100 of FIG. 1. In particular, FIG. 2A illustrates the closure system 100 in an initial configuration. In this initial configuration, the plunger member 130 may be fully retracted relative to the handle member 110, and the anchor portion 152 of the anchor member 150 may be disposed within the inner lumen 140. Advancing the plunger member 130 in a distal direction relative to the handle member 110, the tube set 120, and the inner lumen 140 may deploy the anchor portion 152 of the anchor member 150 from the inner lumen 140, as shown in FIG. 2B. As a result, the anchor portion 152 may move from an initial, contracted configuration to a deployed, expanded configuration. In a further implementation, the plunger member 130 may include two or more plunger components. For example, the plunger member 130 may include a first component configured to deploy and/or retract the anchor member 150 and a second component configured to advance and/or retract the inner lumen 140. In a yet further implementation, the first and second components of the plunger member 130 may be movable with respect to one another.

Thereafter, retracting the plunger member 130 in a proximal direction relative to the handle member 110, the tube set 120, and/or the inner lumen 140 may retract the anchor portion 152 in a proximal direction, as shown in FIG. 2C. As shown in FIG. 2D, advancing the handle member 110 in a distal direction relative to the plunger member 130 may advance the tube set 120 in a distal direction until the distal end 124 of the tube set 120 is proximate the anchor portion 152 of the anchor member 150. As a result, an operator of the closure system 100 may locate, anchor, and/or immobilize the tissue surrounding a body lumen opening between the tube set 120 and anchor portion 152. Thereafter, the operator may deploy a closure element into the body lumen surrounding the body lumen opening to close the body lumen opening.

Reference is now made to FIGS. 3A-3B, which disclose an example anchor member 350 in accordance with implementations of the present disclosure. The example anchor member 350 of this configuration may be functionally similar to the example anchor member 150 previously described above and shown in FIGS. 1-2 in most respects, wherein certain features will not be described in relation to this configuration wherein those components may function in the manner as described above and are hereby incorporated into this additional configuration described below. Like structures and/or components are given like reference numerals. Additionally, the anchor member 350 may incorporate at least one component of the anchor members 450, 550, 650, 750, 850, 950, 1050, 1150, 1250, 1350, 1450, 1550, 1650, 1750, 1950, 2050, and 2150 described in connection with FIGS. 4-21, respectively.

The anchor member 350 may be configured to assist an operator to locate, anchor, immobilize, and/or support a body lumen opening and/or the surrounding tissue of the lumen wall. The anchor member 350 may include an anchor portion 352 and an elongate portion 354. The anchor portion 352 may include any size and/or shape configured to anchor against a surface of a lumen wall or to locate a body lumen opening. For example, the anchor portion may include a plurality of projections 356 configured to engage the tissue of a lumen wall. The projections 356 may be shaped, positioned, and/or oriented in any configuration desired to provide positioning or anchoring support. The anchor portion 352 may include any number of projections 356 desired. In the embodiment shown in FIGS. 3A-3B the anchor portion 352 of the anchor member 350 includes four projections 356, however, the anchor portion 352 may have fewer or more projections 356 than four.

In one embodiment, the projections may extend in a direction or a plane substantially perpendicular to the longitudinal axis of the elongate portion 354. In one configuration, the projections 356 may be rounded. In particular, the projections 356 may be leaf-shaped or pedal-shaped. In a further embodiment, the anchor portion 352 may have a shape substantially similar to a four leaf clover.

The anchor portion 352 may be coupled to the distal end of the elongate portion 354. The elongate portion 354 may include one or more elongate members 358. The elongate member(s) 358 may be configured to advance, retract, position, and/or deploy the anchor portion 352. In particular, the elongate member(s) 358 may be longitudinally rigid or semi-rigid to facilitate advancing or retracting the anchor portion 352. In one embodiment, the elongate member(s) 358 may have a solid configuration such as a nitinol wire or a mandrel. In further embodiments, the elongate member(s) 358 may have a generally tubular configuration.

The anchor portion 352 and/or elongate portion 354 may include any number of materials. In one embodiment, the anchor portion 352 may include the same materials as the elongate portion 354. In a further embodiment, the anchor portion 352 may include different materials than the elongate portion 354.

In one embodiment, the anchor portion 352 and elongate portion 354 may include a single shape memory or superelastic wire forming both the elongate portion 354 and the anchor portion 352. The wire may be set into any shape desired for the elongate portion 354 and anchor portion 352. In particular, the wire may be set in an elongate form for the elongate portion 354 and may be set with a plurality of bights or beds forming the expanded form of the anchor portion 352. As shown in FIGS. 3A-3B, in one configuration, the wire may form a plurality of projections 356.

The anchor portion 352 may be configured to elastically deform to any shape and then return to its expanded shape illustrated FIGS. 3A-3B once released. For example, the anchor portion 352 may be elastically deformed into an elongate and/or contracted configuration and disposed within a lumen. While in this contracted configuration, the anchor portion 352 may store sufficient energy to return to its expanded configuration. Once the anchor portion 352 is deployed from the lumen, the anchor portion 352 may release the stored energy and return to its expanded configuration.

In a further embodiment, the anchor portion 352 of the anchor member 350 may include one or more gripping elements along a proximal surface. The gripping elements may be configured to provide a frictional or immobilizing force on tissue surrounding a body lumen opening. For example, the anchor portion 352 may include a plurality of ridges or teeth along a proximal surface configured to engage and grip or immobilize the tissue surrounding a body lumen opening.

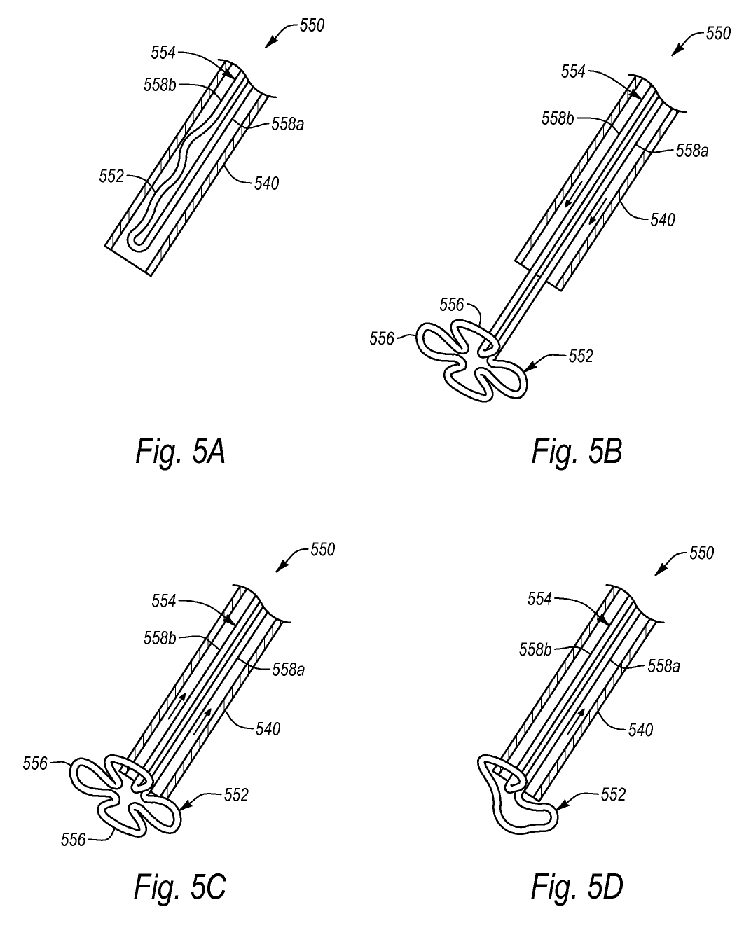

Reference is now made to FIGS. 4A-4B, which illustrate an additional anchor member 450 in accordance with a further embodiment of the present disclosure. The example anchor member 450 of this configuration may be functionally similar to the example anchor members 150 and 350 previously described above and shown in FIGS. 1-3 in most respects, wherein certain features will not be described in relation to this configuration wherein those components may function in the manner as described above and are hereby incorporated into this additional configuration described below. Like structures and/or components are given like reference numerals. Additionally, the anchor member 450 may incorporate at least one component of the anchor members 550, 650, 750, 850, 950, 1050, 1150, 1250, 1350, 1450, 1550, 1650, 1750, 1950, 2050, and 2150 described in connection with FIGS. 5-21, respectively.

In one embodiment, the anchor member 450 may include an anchor portion 452 and an elongate portion 454. The anchor portion 452 may include a plurality of projections 456 extending substantially perpendicular to the longitudinal axis of the elongate portion 454. As shown, the anchor portion 452 may include a FIG.-8 shape having two projections 456. However, the anchor portion 452 may be configured to have any desired shape and/or size having any number of projections.

The elongate portion 454 may include one or more elongate members 458. In one embodiment, the elongate member(s) 458 and anchor portion 452 may be part of a single continuous piece of shape memory or superelastic wire. For example, the wire may extend along the elongate portion 454 and may form the projections 456 of the anchor portion 452 and then may terminate or alternatively extend again along the elongate portion 454. In a further embodiment, portions of the wire may overlap itself or cross over in forming the anchor portion 452. The overlaps or crosses of the wire may provide better resistance against collapse or more support to the anchor portion 452.

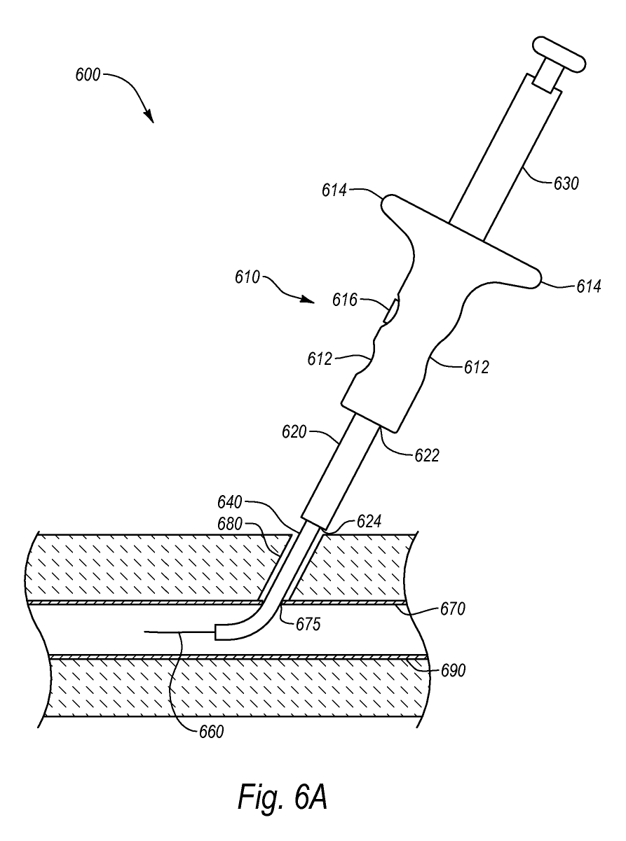

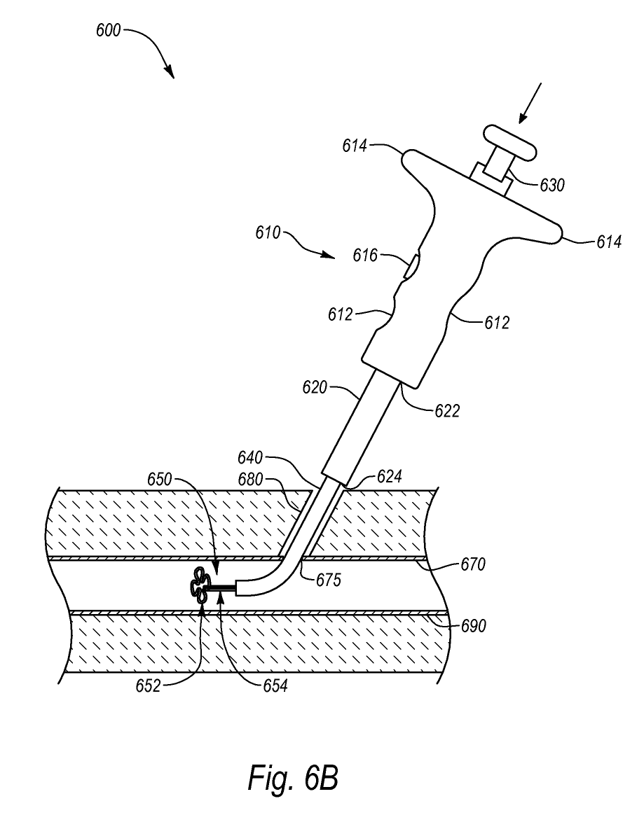

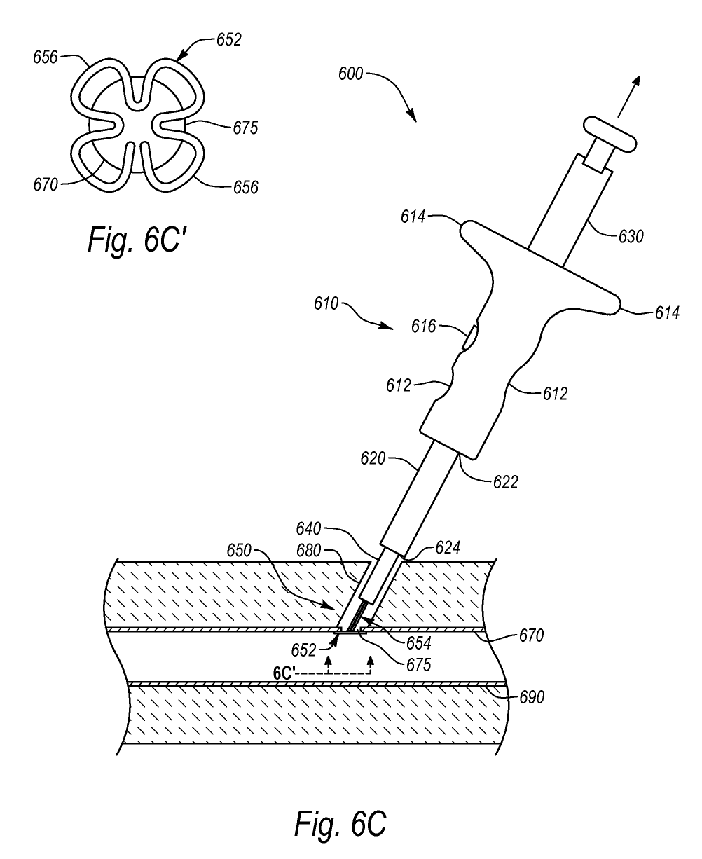

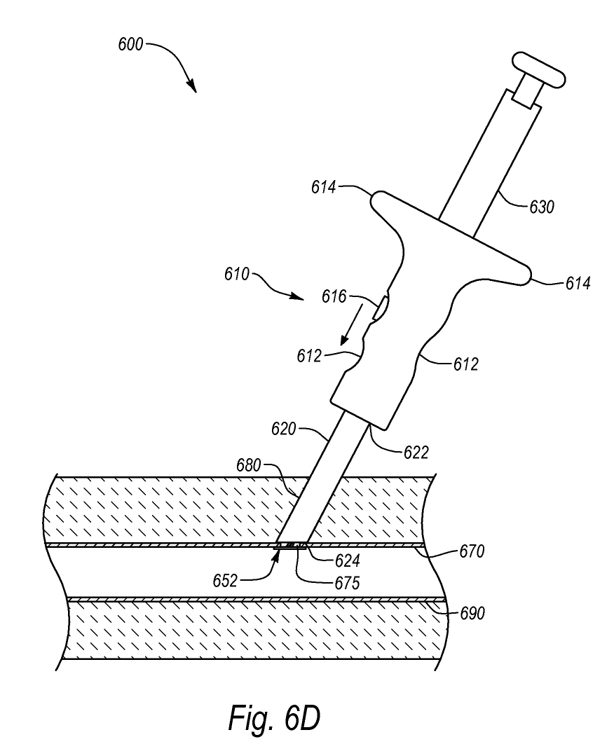

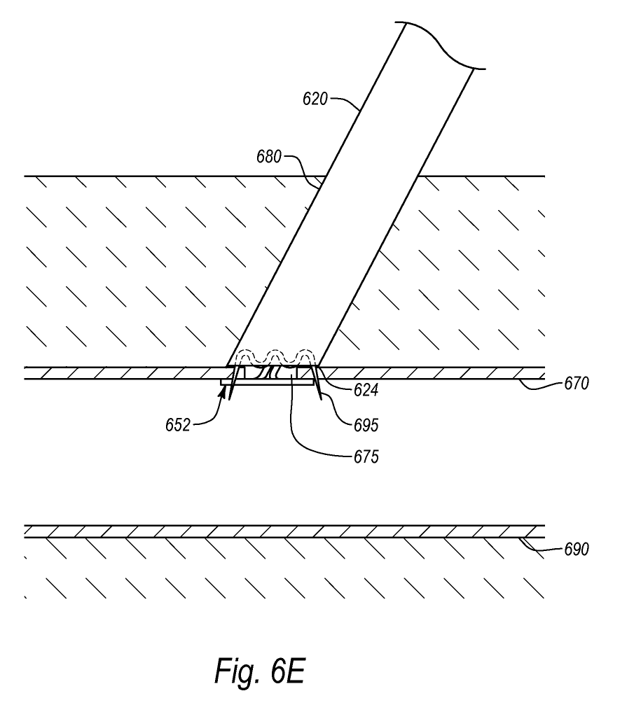

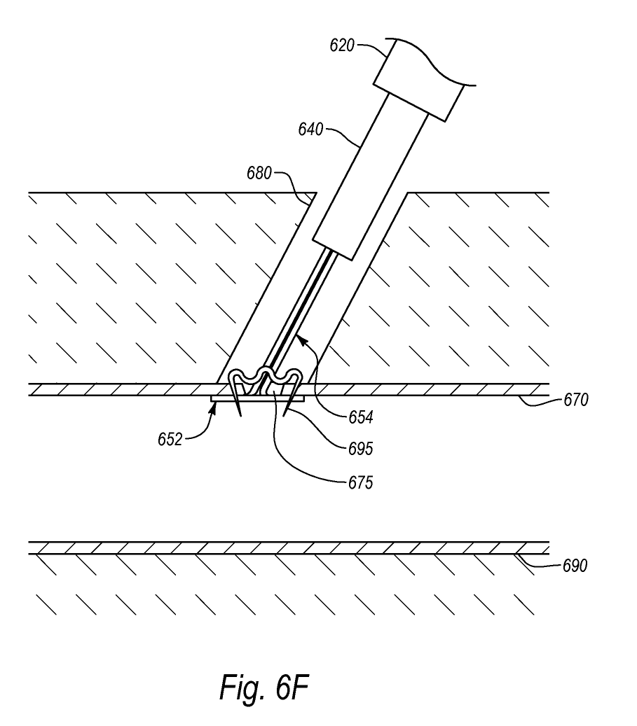

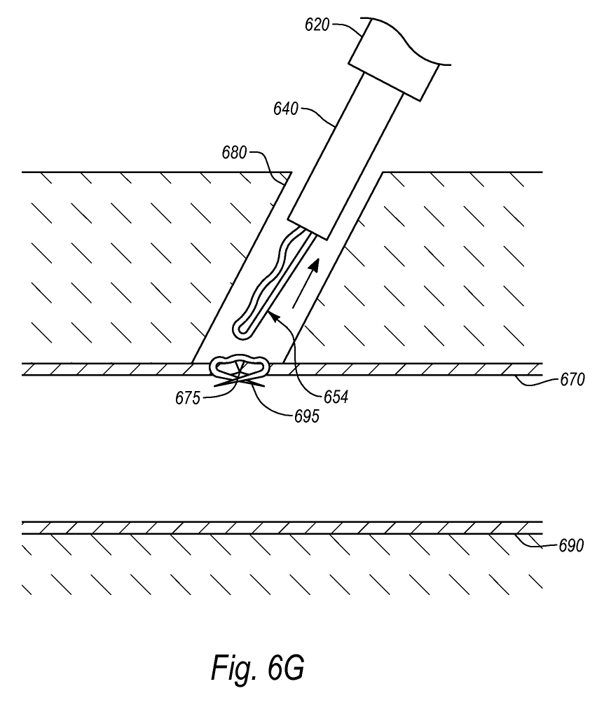

Reference is now made to FIGS. 5A-5D, which illustrate a method of deploying and retracting an anchor member 550. The example anchor member 550 of this configuration may be functionally similar to the example anchor members 150, 350, and 450 previously described above and shown in FIGS. 1-4 in most respects, wherein certain features will not be described in relation to this configuration wherein those components may function in the manner as described above and are hereby incorporated into this additional configuration described below. Like structures and/or components are given like reference numerals. Additionally, the anchor member 550 may incorporate at least one component of the anchor members 650, 750, 850, 950, 1050, 1150, 1250, 1350, 1450, 1550, 1650, 1750, 1950, 2050, and 2150 described in connection with FIGS. 6-21, respectively. Like structures and/or components are given like reference numerals.

In particular, FIG. 5A illustrates the anchor member 550 disposed within a lumen 540 in an initial, contracted configuration. As shown, the anchor member 550 may include an elongate portion 554 and an anchor portion 552. The elongate portion 554 may include a plurality of elongate members 558, such as a first elongate member 558A and a second elongate member 558B.

As shown in FIG. 5B advancing the elongate portion 554, such as one or both of the elongate members 558, in a distal direction relative to the lumen 540 may deploy the anchor portion 552 from the distal end of the lumen 540. As a result, the anchor portion 552 may move from an initial, contracted configuration to a deployed, expanded configuration. In one embodiment, the deployed, expanded configuration may include a plurality of projections 556. In a further embodiment, retracting the elongate portion 554 in a proximal direction may provide an anchoring force. For example, retracting the elongate members 558 may anchor the anchor portion 552 against the distal surface of a lumen wall or any other surface against which the anchor portion 552 is positioned, as shown in FIG. 5C. In one embodiment, retracting both elongate members 558 simultaneously may produce tension or some other force in the anchor portion 552 which may increase the resistance of the anchor portion 552 to contracting. For example, the tension of both elongate members 558 may be simultaneously transferred to the anchor portion 552 thereby creating sufficient tension in the anchor portion 552 to resist movement by the anchor portion 552 away from its expanded configuration. In addition, providing an opposing force against a proximal surface of the anchor portion 552, such as with the lumen wall, may also assist in creating sufficient tension in the anchor portion 552 to resist contraction of the anchor portion 552. In a further implementation, the wires of the anchor portion 552 may overlap or cross over each other in order to increase resistance.

As shown in FIG. 5D, retracting only one elongate member, such as the first elongate member 558A, may lessen the tension in the anchor portion 552, thereby allowing the anchor portion to move from its deployed, expanded configuration to a contracted configuration. As a result, by retracting only the first elongate member 558A, without applying tension to the second elongate member 558B or with applying a distal force to the second elongate member 558B, the anchor portion 552 may contract and be retracted into the lumen 540. In further implementations, by retracting only the second elongate member 558B, without applying tension to the first elongate member 558A or with applying a distal force to the first elongate member 558A, the anchor portion 552 may contract and/or be retracted into the lumen 540.

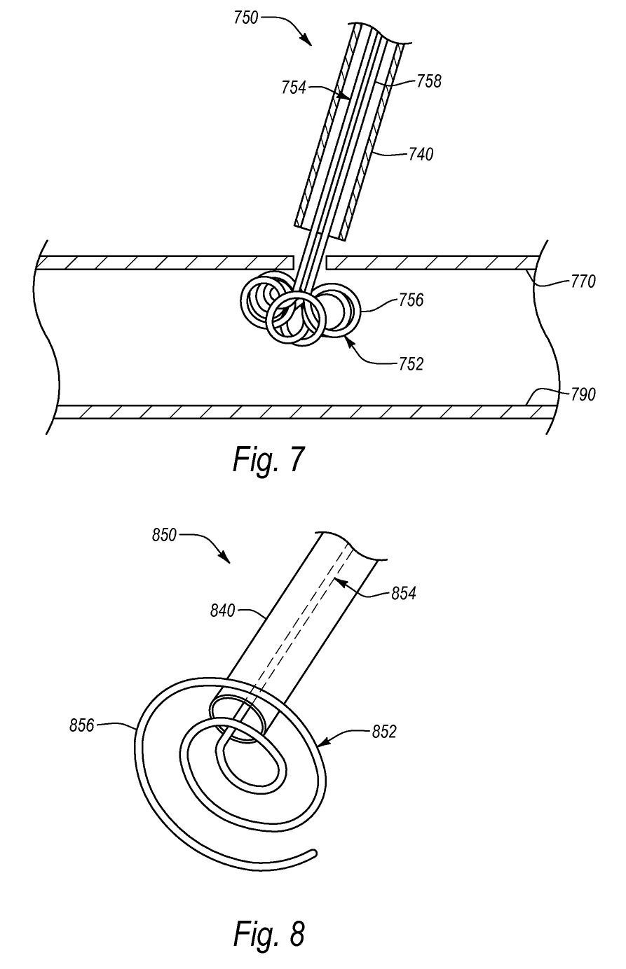

Reference is now made to FIGS. 6A-6G, which illustrate a method of closing a body lumen opening using a closure system 600. The example anchor member 650 of this configuration may be functionally similar to the example anchor members 150, 350, 450, and 550 previously described above and shown in FIGS. 1-5 in most respects, wherein certain features will not be described in relation to this configuration wherein those components may function in the manner as described above and are hereby incorporated into this additional configuration described below. Additionally, the anchor member 650 may incorporate at least one component of the anchor members 750, 850, 950, 1050, 1150, 1250, 1350, 1450, 1550, 1650, 1750, 1950, 2050, and 2150 described in connection with FIGS. 7-21, respectively. Like structures and/or components are given like reference numerals.

As shown in FIG. 6A, the closure system 600 may be at least partially advanced into a body lumen opening. For example, after completing a percutaneous medical procedure, an operator may advance the closure system 600 over a guidewire 660 through a tissue tract 680 and through a body lumen opening 675 in a lumen wall 670. In particular, the operator may advance the closure system 600 until the inner lumen 640 of the closure system 600 extends at least partially into the body lumen 690. Once the closure system 600 has been advanced at least partially into the body lumen 690 the operator may then retract the guidewire 660 from the body lumen 690.

As shown in FIG. 6B, once the closure system 600 is advanced into the body lumen 690, the operator may deploy the anchor member 650 into the body lumen 690. As explained in more detail above, the operator may deploy the anchor member 650 by advancing the plunger member 630 and/or elongate portion 654 in a distal direction relative to the handle member 610, the tube set 620, and the inner lumen 640. Once deployed from the inner lumen, the anchor portion 652 of the anchor member 650 may move from an initial, contracted configuration to a deployed, expanded configuration. As shown in FIG. 6C, once the anchor portion 652 of the anchor member 650 has been deployed within the body lumen 690, the operator may retract the plunger member 630 and/or closure system 600 to position the anchor portion 652 of the anchor member 650 against the distal surface of the lumen wall 670 proximate the body lumen opening as also shown in FIG. 6C. In particular, the operator may retract the plunger member 630 and/or closure system 600 until she feels the anchoring force or resistance from the anchor portion 652 of the anchor member 650 against the distal surface of the lumen wall 670 thereby locating the body lumen opening 675 and anchoring or securing the tissue surrounding the body lumen opening 675. As shown, the anchor portion 652 may include a plurality of projections 656 which engage and anchor the tissue of the lumen wall 670. In particular, the projections 656 may extend in a direction substantially perpendicular to the longitudinal axis of the elongate portion 654, the tube set 620, and/or inner lumen 640.

Once the anchor portion 652 has located the body lumen opening 675 and/or anchored or secured the tissue surrounding the body lumen opening 675, the operator may advance the handle member 610 in a distal direction relative to the plunger member 630 in order to advance the tube set 620 in a distal direction relative to the anchor portion 652. In particular, the operator may advance the handle member 610 and/or tube set 620 until the distal end 624 of the tube set 620 engages the proximal surface of the lumen wall 670 proximate or surrounding the lumen opening. As a result, in one embodiment, by advancing the tube set 620 in a distal direction and/or retracting the anchor portion 652 in a proximal direction, the operator may sandwich the tissue of the lumen wall 670 surrounding the body lumen opening 675 between the tube set 620 and the anchor portion 652. Accordingly, the operator may thereby engage and/or at least partially immobilize the tissue surrounding the body lumen opening 675. This may facilitate the successful deployment of a closure element 695 into the tissue surrounding the body lumen opening 675, thereby, facilitating the closure of the body lumen opening 675. In particular, the tube set 620 and the anchor portion 652 may hold the tissue in place while a closure element is deployed into the tissue. Therefore, as shown in FIG. 6E the operator may then deploy a closure element 695 into the tissue surrounding the body lumen opening. In one embodiment, the operator may depress the button 616 to eject or deploy the closure element 695 into the lumen wall 670. In particular, the closure element 695 may be deployed from an initial, open configuration to a deployed, closed configuration, thereby, engaging and bringing the tissue surrounding the body lumen opening 675 together to close the body lumen opening 675. The closure element 695 may include any device configured to close a body lumen opening 675. For example, the closure element 695 may include a staple, a clip, other similar devices, or combinations thereof.

As shown in FIG. 6F, once the closure element 695 has been deployed, the handle member 610, tube set 620, and/or inner lumen 640 may be retracted out of and/or away from the body lumen 690 and tissue tract 680, as shown in FIG. 6F. Thereafter, the anchor member 650 may be retracted by retracting the elongate portion 654 in a proximal direction. For example, in one embodiment the anchor portion 652 may be pulled through the closure element 695. The closure element 695 may have superelastic properties to facilitate the withdrawal of the anchor portion 652 through the closure element 695. For example, the closure element may at least partially expand to facilitate the withdrawal of the anchor portion 652 and then return to a contracted position to close the body lumen opening 675. Accordingly, by following one or more of the acts disclosed in FIGS. 6A-6G, an operator may efficiently close a body lumen opening 675 with a greater amount of flexibility and control.

In one embodiment, the inner lumen 640 can be held in place against the outer surface of the body lumen while the anchor member 650 is retracted. Holding the inner lumen 640 may provide sufficient force to allow the anchor member and more particularly the anchor portion 652 to deform into the pre-deployment state inside of the inner lumen 640. As previously stated, this may be achieved by retracting a single elongate member. This may ensure that the closure element does not become dislodged as the anchor portion 652 is withdrawn and contracted. In further embodiments, the anchor wire may be substantially smaller than the closure element. As a result, pulling the anchor portion 652 through the closure element may not affect the positioning of the closure element since the closure element anchors in the tissue by design. In one implementation, the wire of the anchor portion 652 may be superelastic with a diameter small enough to not require substantial force to collapse the anchor portion 652 and pull it through the deployed closure element. For example, the anchor wire may have a diameter of around 0.005-0.007″.

In one configuration, the anchor, closure element, and/or other aspects or components of the closure system disclosed herein can be made of a single material or of multiple materials. This can include a metal primary material and polymer/drug topcoat or a different metal top layer. The multiple layers can be resiliently flexible materials or rigid and inflexible materials, and selected combinations thereof. The use of resiliently flexible materials can provide force-absorbing characteristics, which can also be beneficial for absorbing stress and strains, which may inhibit crack formation at high stress zones. Also, the multiple layers can be useful for applying radiopaque materials. For example, types of materials that are used to make a closure element can be selected so that the closure element is capable of being in a first orientation (e.g., delivery orientation) during placement and capable of transforming to a second orientation (e.g., deploying orientation) when deployed to close the opening in a lumen.

Embodiments of the anchor, closure element and the like can include a material made from any of a variety of known suitable biocompatible materials, such as a biocompatible shape memory material (SMM). For example, the SMM can be shaped in a manner that allows for a delivery orientation while within the tube set, but can automatically retain the memory shape of the closure element once deployed into the tissue to close the opening. SMMs have a shape memory effect in which they can be made to remember a particular shape. Once a shape has been remembered, the SMM may be bent out of shape or deformed and then returned to its original shape by unloading from strain or heating. Typically, SMMs can be shape memory alloys (SMA) comprised of metal alloys, or shape memory plastics (SMP) comprised of polymers. The materials can also be referred to as being superelastic.

Usually, an SMA can have an initial shape that can then be configured into a memory shape by heating the SMA and conforming the SMA into the desired memory shape. After the SMA is cooled, the desired memory shape can be retained. This allows for the SMA to be bent, straightened, twisted, compacted, and placed into various contortions by the application of requisite forces; however, after the forces are released, the SMA can be capable of returning to the memory shape. The main types of SMAs are as follows: copper-zinc-aluminum; copper-aluminum-nickel; nickel-titanium (NiTi) alloys known as nitinol; nickel-titanium platinum; nickel-titanium palladium; and cobalt-chromium-nickel alloys or cobalt-chromium-nickel-molybdenum alloys known as elgiloy alloys. The temperatures at which the SMA changes its crystallographic structure are characteristic of the alloy, and can be tuned by varying the elemental ratios or by the conditions of manufacture. This can be used to tune the closure element so that it reverts to the memory shape to close the arteriotomy when deployed at body temperature and when being released from the tube set.

For example, the primary material of a closure element can be of a NiTi alloy that forms superelastic nitinol. In the present case, nitinol materials can be trained to remember a certain shape, retained within the tube set, and then deployed from the tube set so that the tines penetrate the tissue as it returns to its trained shape and closes the opening. Also, additional materials can be added to the nitinol depending on the desired characteristic. The alloy may be utilized having linear elastic properties or non-linear elastic properties.

An SMP is a shape-shifting plastic that can be fashioned into a closure element in accordance with the present disclosure. Also, it can be beneficial to include at least one layer of an SMA and at least one layer of an SMP to form a multilayered body; however, any appropriate combination of materials can be used to form a multilayered device. When an SMP encounters a temperature above the lowest melting point of the individual polymers, the blend makes a transition to a rubbery state. The elastic modulus can change more than two orders of magnitude across the transition temperature (Ttr). As such, an SMP can be formed into a desired shape of an endoprosthesis by heating it above the Ttr, fixing the SMP into the new shape, and cooling the material below Ttr. The SMP can then be arranged into a temporary shape by force and then resume the memory shape once the force has been released. Examples of SMPs include, but are not limited to, biodegradable polymers, such as oligo(ε-caprolactone)diol, oligo(ρ-dioxanone)diol, and non-biodegradable polymers such as, polynorborene, polyisoprene, styrene butadiene, polyurethane-based materials, vinyl acetate-polyester-based compounds, and others yet to be determined. As such, any SMP can be used in accordance with the present disclosure.

An anchor, closure element and the like may have at least one layer made of an SMM or suitable superelastic material and other suitable layers can be compressed or restrained in its delivery configuration within the garage tube or inner lumen, and then deployed into the tissue so that it transforms to the trained shape. For example, a closure element transitions to close the opening in the body lumen while an anchor may expand to anchor the closure system.

Also, the anchor, closure element, or other aspects or components of the closure system can be comprised of a variety of known suitable deformable materials, including stainless steel, silver, platinum, tantalum, palladium, nickel, titanium, nitinol, nitinol having tertiary materials (U.S. 2005/0038500, which is incorporated herein by reference, in its entirety), niobium-tantalum alloy optionally doped with a tertiary material (U.S. 2004/0158309, 2007/0276488, and 2008/0312740, which are each incorporated herein by reference, in their entireties) cobalt-chromium alloys, or other known biocompatible materials. Such biocompatible materials can include a suitable biocompatible polymer in addition to or in place of a suitable metal. The polymeric closure element can include biodegradable or bioabsorbable materials, which can be either plastically deformable or capable of being set in the deployed configuration.

In one embodiment, the closure element or anchor may be made from a superelastic alloy such as nickel-titanium or nitinol, and includes a ternary element selected from the group of chemical elements consisting of iridium, platinum, gold, rhenium, tungsten, palladium, rhodium, tantalum, silver, ruthenium, or hafnium. The added ternary element improves the radiopacity of the nitinol closure element. The nitinol closure element has improved radiopacity yet retains its superelastic and shape memory behavior and further maintains a thin body thickness for high flexibility.

In one embodiment, the anchor or closure element can be made at least in part of a high strength, low modulus metal alloy comprising Niobium, Tantalum, and at least one element selected from the group consisting of Zirconium, Tungsten, and Molybdenum.

In further embodiments, the closure element or anchor can be made from or be coated with a biocompatible polymer. Examples of such biocompatible polymeric materials can include hydrophilic polymer, hydrophobic polymer biodegradable polymers, bioabsorbable polymers, and monomers thereof. Examples of such polymers can include nylons, poly(alpha-hydroxy esters), polylactic acids, polylactides, poly-L-lactide, poly-DL-lactide, poly-L-lactide-co-DL-lactide, polyglycolic acids, polyglycolide, polylactic-co-glycolic acids, polyglycolide-co-lactide, polyglycolide-co-DL-lactide, polyglycolide-co-L-lactide, polyanhydrides, polyanhydride-co-imides, polyesters, polyorthoesters, polycaprolactones, polyesters, polyanydrides, polyphosphazenes, polyester amides, polyester urethanes, polycarbonates, polytrimethylene carbonates, polyglycolide-co-trimethylene carbonates, poly(PBA-carbonates), polyfumarates, polypropylene fumarate, poly(p-dioxanone), polyhydroxyalkanoates, polyamino acids, poly-L-tyrosines, poly(beta-hydroxybutyrate), polyhydroxybutyrate-hydroxyvaleric acids, polyethylenes, polypropylenes, polyaliphatics, polyvinylalcohols, polyvinylacetates, hydrophobic/hydrophilic copolymers, alkylvinylalcohol copolymers, ethylenevinylalcohol copolymers (EVAL), propylenevinylalcohol copolymers, polyvinylpyrrolidone (PVP), combinations thereof, polymers having monomers thereof, or the like.

Reference is now made to FIG. 7, which discloses an example anchor member 750 in accordance with implementations of the present disclosure. The example anchor member 750 of this configuration may be functionally similar to the example anchor members 150, 350, 450, 550, and 650 previously described above and shown in FIGS. 1-6 in most respects, wherein certain features will not be described in relation to this configuration wherein those components may function in the manner as described above and are hereby incorporated into this additional configuration described below. Like structures and/or components are given like reference numerals. Additionally, the anchor member 750 may incorporate at least one component of the anchor members 850, 950, 1050, 1150, 1250, 1350, 1450, 1550, 1650, 1750, 1950, 2050, and 2150 described in connection with FIGS. 8-21, respectively.

The anchor member 750 may include an anchor portion 752 and an elongate portion 754. The anchor portion 752 may have a deployed, expanded configuration comprising a plurality of projections 756. The projections 756 may include any of a number of bights and loops. In one embodiment, the projections 756 may be irregularly shaped. In a further embodiment, the projections 756 may be irregularly distributed and/or positioned. In particular, in one embodiment, the anchor portion 752 may be similar to an aneurysm coil. In a further embodiment, the anchor portion 752 may comprise a configuration similar to a bird's nest.

Prior to deployment, the anchor portion 752 may be disposed within the delivery lumen 740 in an initial contracted configuration. For example, the anchor portion 752 may be elongated and/or contracted into the initial configuration and disposed within the delivery lumen 740. The anchor portion 752 may then be deployed from the delivery lumen 740 at which time it may move to an expanded or deployed configuration, as shown in FIG. 7.

The elongate portion 754 may facilitate the deployment, positioning, anchoring, retention, and/or retraction of the anchor portion 752. For example, the elongate portion 754 may be coupled to the anchor portion 752 and may extend away from the anchor portion 752 in a proximal direction where it can be manipulated and/or controlled by an operator. In one embodiment, the elongate portion 754 may include one or more elongate members 758.

In a further embodiment, an operator may deploy the anchor portion 752 by advancing the elongate member(s) 758 of the elongate portion 754 in a distal direction relative to the delivery lumen 740. The operator may then engage the anchor portion 752 against a surface, such as a lumen wall 770, by applying tension to the elongate member(s) 758. In one configuration, by applying tension to both the first elongate member 758a and the second elongate member 758b an operator may create sufficient tension in the anchor portion 752 to prevent unfolding or contraction of the anchor portion 752. This may be aided by opposing force created by the delivery lumen 740 or lumen wall 770.

For example, the operator may retract the anchor member 750 in a proximal direction to engage the distal surface of a lumen wall 770 with the anchor portion 752. The operator may then advance the delivery lumen in a distal direction to engage a proximal surface of a lumen wall 770, thereby immobilizing the lumen wall 770 and/or providing an opposing force to the anchor portion 752. Once an operator desires to remove the anchor portion 752 from the body lumen 790, the operator may apply tension to the first elongate member 758a but may immobilize the second elongate member 758b or advance the second elongate member 750b in a distal direction. This may facilitate the uncoiling, elongation, and/or contraction of the anchor portion 752, thereby, facilitating removal of the anchor portion 752 from the body lumen 790.

The anchor member 750 may comprise any number of different materials. In one embodiment, the elongate portion 754 and/or the anchor portion 752 of the anchor member 750 may comprise a shape memory or superelastic material. For example, the elongate portion 754 and anchor portion 752 may comprise any number of shape memory alloys. In a further embodiment, the anchor member 750 may comprise a single shape memory wire forming both the elongate portion 754 and the anchor portion 752. In yet further embodiments, the elongate portion 754 may comprise a different material than the anchor portion 752. For example, the elongate portion 754 may comprise a tubular member, mandrel, or wire to facilitate the deployment and retraction of the anchor portion 752 and the anchor portion 752 may comprise a bioerodible, bioabsorbable, bioresorbable, and/or biodegradable material.

In one embodiment, the elongate portion 754 may be detachable from the anchor portion 752. For example, the elongate portion may be trimmed once a medical procedure is complete in order to retract the elongate portion leaving it in place or releasing the anchor portion 752 into the body lumen 790.

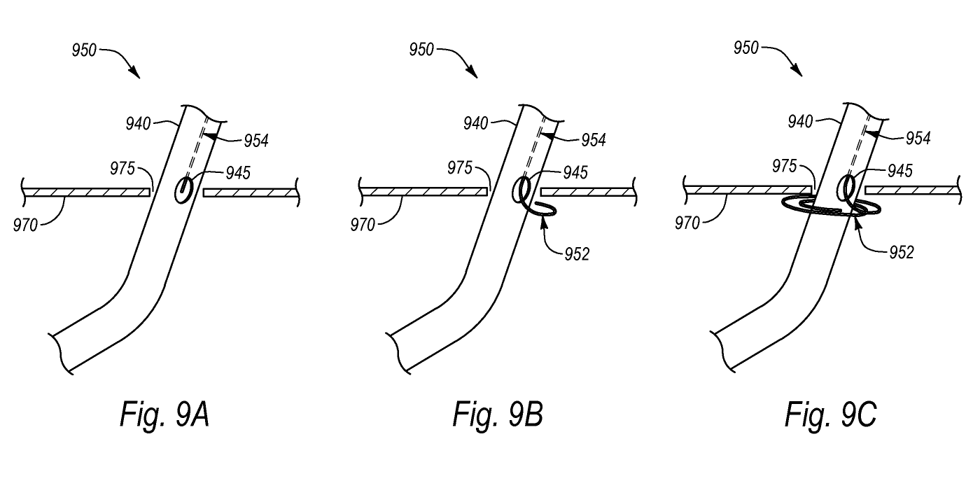

Reference is now made to FIG. 8 which illustrates another example anchor member 850 in accordance with the present disclosure. The anchor member 850 may be similar in many respects to the anchor members 150, 350, 450, 550, 650, and 750 previously described above and shown in FIGS. 1-7 in most respects, wherein certain features will not be described in relation to this configuration wherein those components may function in the manner as described above and are hereby incorporated into this additional configuration described below. Like structures and/or components are given like reference numerals. Additionally, the anchor member 850 may incorporate at least one component of the anchor members 950, 1050, 1150, 1250, 1350, 1450, 1550, 1650, 1750, 1950, 2050, and 2150 described in connection with FIGS. 9-21, respectively.

As shown in FIG. 8, the anchor member 850 may include an anchor portion 852 and an elongate portion 854. The anchor member 850 may also be partially disposed within a delivery lumen 840. The anchor portion 852 may comprise one or more spirals 856. Each spiral may extend circularly about the longitudinal axis of the elongate portion any number of times. In addition, the anchor portion 852 may extend incrementally outward with each spiral 856. In one embodiment, the anchor portion 852 may rotate around two or more times.

Initially, the anchor portion 852 may be disposed at least partially within the delivery lumen 840. For example, in one embodiment, the anchor portion 852 may be disposed within the delivery lumen 840 in an initial configuration, wherein the spirals 856 of the anchor portion 852 may be elongated and/or contracted to facilitate disposal within the delivery lumen 840. As a result the delivery lumen 840 may deliver the anchor member 850 proximate to or within a body lumen.

In one embodiment, an operator may deploy the anchor portion 852 from the delivery lumen 840 by advancing the elongate portion 854 in a distal direction relative to the delivery lumen 840. The anchor portion 852 may then be deployed from the distal end of the delivery lumen by extending out and expanding into its expanded configuration as shown in FIG. 8. Although, as shown in FIG. 8, the anchor portion 852 extends from a distal opening in the delivery lumen 840. In a further configuration, the anchor portion may extend out of a lateral opening in the delivery lumen 840, as shown in FIGS. 9A through 9C.

Once deployed, an operator may utilize the anchor portion 852 to anchor and/or locate the tissue surrounding a body lumen opening to facilitate completion of a medical procedure, such as closure of the body lumen opening. Once the medical procedure is complete, the operator may remove the anchor portion 852 out of and away from the body lumen by retracting the elongate member 854 in a proximal direction relative to the body lumen.

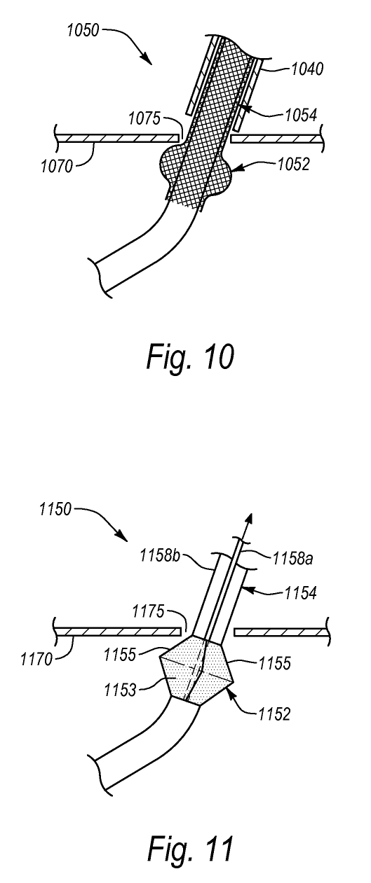

Reference is now made to FIGS. 9A-9C, which disclose deployment of an example anchor member 950. The anchor member 950 may be similar in many respects to the anchor members 150, 350, 450, 550, 650, 750, and 850 previously described above and shown in FIGS. 1-8 in most respects, wherein certain features will not be described in relation to this configuration wherein those components may function in the manner as described above and are hereby incorporated into this additional configuration described below. Like structures and/or components are given like reference numerals. Additionally, the anchor member 950 may incorporate at least one component of the anchor members 1050, 1150, 1250, 1350, 1450, 1550, 1650, 1750, 1950, 2050, and 2150 described in connection with FIGS. 10-21, respectively.

As shown, the anchor member 950 may be similar to the anchor member 850 illustrated in FIG. 8 and discussed in more detail above. In one embodiment, the anchor member 950 may be deployed from a lateral opening 945 in a delivery lumen 940. As shown in FIG. 9A, the delivery lumen 940 may be advanced at least partially into a body lumen opening 975 in a lumen wall 970. In particular, the delivery lumen 940 may be advanced through the body lumen opening 975 until the lateral opening 945 in the delivery lumen 940 is positioned distal of the lumen wall 970.

Thereafter, as shown in FIG. 9B, an operator may begin to deploy the anchor portion 952 of the anchor member 950 from the lateral opening 945 of the delivery lumen 940. In particular, the operator may advance the elongate portion 954 in a distal direction relative to the delivery lumen 940. The anchor portion 952 may extend out of the lateral opening 945 and begin to spiral around the delivery lumen 940 as the operator advances the elongate portion 954.

As shown in FIG. 9C, once the anchor portion 952 is fully deployed the operator may anchor the anchor portion 952 against the distal surface of the lumen wall 970 to assist in the completion of a medical procedure, such as closure of the body lumen opening 975. Once the desired medical procedure is complete, the operator may then retract the anchor portion back into the delivery lumen 940 through the lateral opening 945 by retracting the elongate portion 954 in a proximal direction relative to the delivery lumen 940. Thereafter, the operator may remove the delivery lumen 940 from the opening 975 in the lumen wall 970.

Reference is now made to FIGS. 10, which illustrate additional example anchor member 1050 in accordance with the present disclosure. The anchor member 1050 may be similar in many respects to the anchor members 150, 350, 450, 550, 650, 750, 850, and 950 previously described above and shown in FIGS. 1-9 in most respects, wherein certain features will not be described in relation to this configuration wherein those components may function in the manner as described above and are hereby incorporated into this additional configuration described below. Like structures and/or components are given like reference numerals. Additionally, the anchor member 1050 may incorporate at least one component of the anchor members 1150, 1250, 1350, 1450, 1550, 1650, 1750, 1950, 2050, and 2150 described in connection with FIGS. 11-21, respectively.

The anchor member 1050 may include an anchor portion 1052 and an elongate portion 1054. In one embodiment, the anchor member 1050 may comprise a mesh material with an expandable portion forming the anchor portion 1052. The anchor member 1050 may be disposed initially in a delivery configuration within a delivery lumen 1040. The delivery lumen 1040 and anchor member 1050 may be inserted through an opening 1075 in a lumen wall 1070. The delivery lumen 1040 may be retracted to expose the anchor portion 1052, which may then self expand or be expanded by a user. The user may then retract the anchor member 1050 to locate and/or anchor the opening 1075 in the lumen wall 1070 to assist in completion of a medical procedure.

Once a desired medical procedure is complete, the user may return the anchor portion 1052 to its contracted configuration. This may be accomplished by advancing the delivery lumen 1040 in a distal direction relative to the anchor portion 1052 to contract the anchor portion 1052 and recapture the anchor portion 1052 within the delivery lumen 1040. Thereafter, the user may retract the anchor member 1050 and delivery lumen 1040 out of and away from the opening 1075 in the lumen wall 1070.

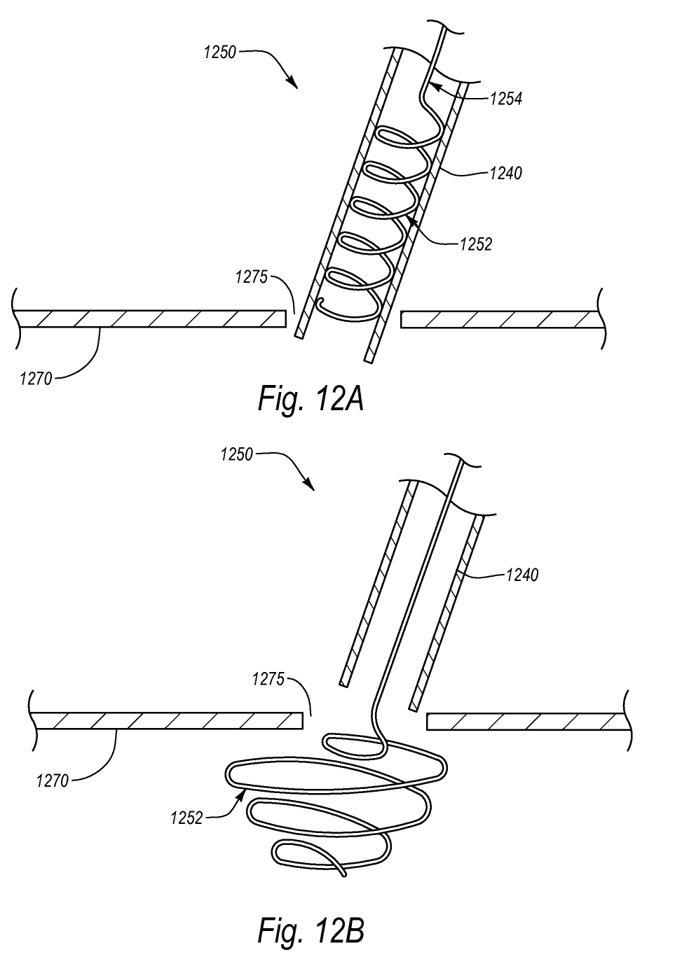

Reference is now made to FIGS. 11, which illustrate additional example anchor member 1150 in accordance with the present disclosure. The anchor member 1150 may be similar in many respects to the anchor members 150, 350, 450, 550, 650, 750, 850, 950, and 1050 previously described above and shown in FIGS. 1-10 in most respects, wherein certain features will not be described in relation to this configuration wherein those components may function in the manner as described above and are hereby incorporated into this additional configuration described below. Like structures and/or components are given like reference numerals. Additionally, the anchor member 1150 may incorporate at least one component of the anchor members 1250, 1350, 1450, 1550, 1650, 1750, 1950, 2050, and 2150 described in connection with FIGS. 12-21, respectively.

The anchor member 1150 may include an anchor portion 1152 and an elongate portion 1154. In one embodiment, the anchor portion 1152 may comprise an expandable membrane 1153. The anchor portion 1152 may also include one or more hinged struts 1155. The anchor portion 1152 may expand and contract as each strut 1155 moves from an elongate configuration to a bent configuration and back to an elongate configuration. Once expanded, an operator may retract the anchor member 1150 in a proximal direction to position the anchor portion 1152 against the distal surface of the lumen wall 1170. The membrane 1153 may assist in providing temporary closure of the opening 1175 during completion of a medical procedure.

In one embodiment, the anchor portion 1152 may be self-expanding. In a further embodiment, the expansion and contraction of the anchor portion 1152 may be facilitated by the elongate portion 1154. For example, the elongate portion 1154 may include a first elongate member 1158a and a second elongate member 1158b. In one embodiment, the first elongate member 1158a may include a mandrel or push/pull wire connected to a distal end of the anchor portion 1152. In a further embodiment, the second elongate member 1158b may be generally tubular and connected to a proximal end of the anchor portion 1152. In addition, the first elongate member 1158a may be disposed at least partially through the second elongate member 1158b. As a result, an operator may expand and contract the anchor portion 1152 using relative movement between the first elongate member 1158a and second elongate member 1158b. For example, the operator may expand the anchor portion 1152 by retracting the first elongate member 1158a in a proximal direction relative to the second elongate member 1158b. Thereafter, the operator may contract the anchor portion 1152 by advancing the first elongate member 1158a relative to the second elongate member 1158b.

As a result, the operator may expand the anchor portion 1152 to use the anchor portion 1152 to locate and/or anchor the opening 1175 in the lumen wall 1170 to assist in completion of a medical procedure, such as closure of the opening. Once the medical procedure is complete, the operator may contract the anchor portion 1152 and retract the anchor portion 1152 out of and away from the lumen wall 1170.

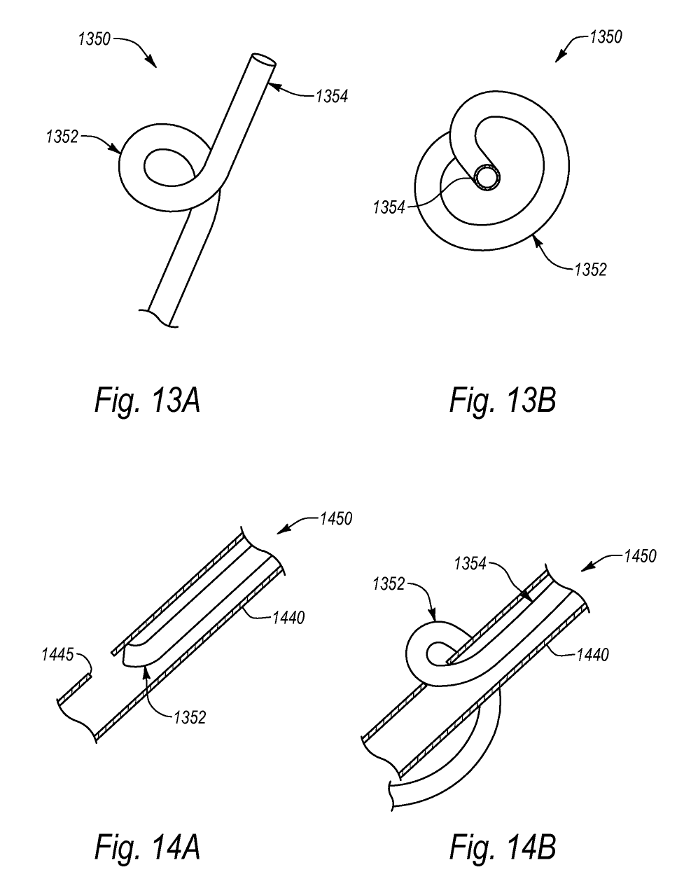

Reference is now made to FIGS. 12, which illustrate additional example anchor member 1250 in accordance with the present disclosure. The anchor member 1250 may be similar in many respects to the anchor members 150, 350, 450, 550, 650, 750, 850, 950, 1050, and 1150 previously described above and shown in FIGS. 1-11 in most respects, wherein certain features will not be described in relation to this configuration wherein those components may function in the manner as described above and are hereby incorporated into this additional configuration described below. Like structures and/or components are given like reference numerals. Additionally, the anchor member 1250 may incorporate at least one component of the anchor members 1350, 1450, 1550, 1650, 1750, 1950, 2050, and 2150 described in connection with FIGS. 13-21, respectively.

As shown, the anchor member 1250 may include an anchor portion 1252 and an elongate portion 1254. In one embodiment, the anchor member may be delivered to or through a body lumen opening 1275 in a lumen wall 1270 using a delivery lumen 1240. For example, the anchor member 1250 may be disposed within the delivery lumen 1240 in an initial contracted configuration. In one embodiment, the anchor portion 1252 may comprise a self-expanding coil. For example, the anchor portion 1252 may coil around the inside of the delivery lumen 1240 in an initial contracted configuration. In addition, the anchor portion 1252 may be configured to expand, through self-expansion or by physical force, to an expanded configuration once deployed from the delivery lumen 1240, as shown in FIG. 12B.

An operator may anchor the expanded anchor portion 1252 against a distal surface of the lumen wall 1270 to anchor and/or locate the tissue surrounding the body lumen opening 1275 to facilitate completion of a medical procedure. Once the medical procedure is complete, the operator may retract the anchor portion 1252 into the delivery lumen 1240 by retracting the elongate portion 1254 in a proximal direction relative to the delivery lumen 1240. Once the anchor portion 1252 is retracted into the delivery lumen 1240, the operator may retract the delivery lumen 1240 out of and away from the body lumen opening 1275.

Reference is now made to FIGS. 13A-13B, which illustrates an additional example anchor member 1350 in accordance with the present disclosure. The anchor member 1350 may be similar in many respects to the anchor members 150, 350, 450, 550, 650, 750, 850, 950, 1050, 1150, and 1250 previously described above and shown in FIGS. 1-12 in most respects, wherein certain features will not be described in relation to this configuration wherein those components may function in the manner as described above and are hereby incorporated into this additional configuration described below. Like structures and/or components are given like reference numerals. Additionally, the anchor member 1350 may incorporate at least one component of the anchor members 1450, 1550, 1650, 1750, 1950, 2050, and 2150 described in connection with FIGS. 14-21, respectively.

FIG. 13A illustrates a perspective view of the anchor member 1350 and FIG. 13B illustrates an end view of the anchor member 1350. In one embodiment, the anchor member 1350 may be generally tubular in shape. As a result, the anchor member 1350 may be advanced over a guidewire or other device. The anchor member 1350 may include an elongate portion 1354 and an anchor portion 1342. In one embodiment, the anchor member 1350 may comprise an elongate and/or tubular member with a straight portion forming the elongate portion 1354 and a looped portion forming the anchor portion 1352. The anchor member 1350 may be formed using any rigid or semi-rigid materials. A user may advance the anchor member 1350 through an opening in a lumen wall by inserting the anchor member 1350 through the opening and then rotating the anchor member 1350 as it is advanced through the opening in the lumen wall in order to pass the anchor portion 1352 through the opening in the lumen wall. The user may then use the anchor member 1350 to locate and/or anchor a device relative to the opening by retracting the anchor portion 1352 against a distal surface of the lumen wall without rotating the anchor member 1350, thereby locating the opening and preventing the anchor portion 1352 from passing through the opening.

In a further embodiment, the anchor portion 1352 of the anchor member 1350 may include one or more shape memory materials and may be configured to move superelastically between a contracted elongate configuration to the expanded configuration shown in FIGS. 13A-13B. For example, a user can dispose the anchor member 1350 through a body lumen opening with the anchor portion 1352 being contracted/elongated. Thereafter, the anchor 1352 may superelastically expand to an expanded configuration so that the user may utilize the anchor portion to effectively located and anchor the body lumen opening. Once a desired medical procedure is complete, the user may return the anchor portion 1352 to a contracted configuration and then retract the anchor member 1350 out of and away from the body lumen opening.

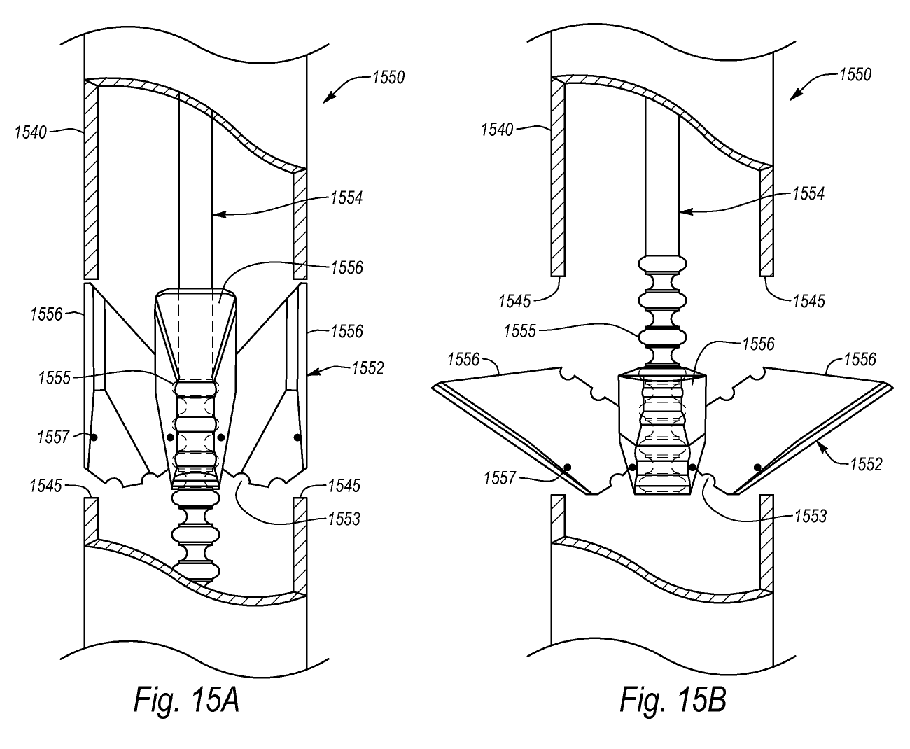

Reference is now made to FIGS. 14A-14B, which illustrates an additional example anchor member 1450 in accordance with the present disclosure. The anchor member 1450 may be similar in many respects to the anchor members 150, 350, 450, 550, 650, 750, 850, 950, 1050, 1150, 1250, and 1350 previously described above and shown in FIGS. 1-13 in most respects, wherein certain features will not be described in relation to this configuration wherein those components may function in the manner as described above and are hereby incorporated into this additional configuration described below. Like structures and/or components are given like reference numerals. Additionally, the anchor member 1450 may incorporate at least one component of the anchor members 1550, 1650, 1750, 1950, 2050, and 2150 described in connection with FIGS. 15-21, respectively.

As shown, the anchor member 1450 may include an anchor portion 1452 and an elongate portion 1454. In one embodiment, the anchor member 1450 may be similar to the anchor member 1350 illustrated in FIGS. 13A-13B and described in more detail above. The anchor member 1450 may be configured to move between a contracted configuration and an expanded configuration. FIG. 14A illustrates a perspective view of the anchor member 1450 disposed in a contracted configuration within a delivery lumen 1440. FIG. 14B illustrates a perspective view of the anchor member 1450 in an expanded configuration.

The delivery lumen 1440 may be advanced at least partially into a body lumen opening to deliver the anchor member 1450 in its initial contracted configuration, as illustrated in FIG. 14A. In particular, the delivery lumen 1440 may be advanced through the body lumen opening until the lateral opening 1445 in the delivery lumen 1440 is positioned distal of the lumen wall.

Thereafter, as shown in FIG. 14B, an operator may deploy the anchor portion 1452 of the anchor member 1450 from the lateral opening 1445 of the delivery lumen 1440. In particular, the operator may advance the elongate portion 1454 in a distal direction relative to the delivery lumen 1440. The anchor portion 1452 may extend out of the lateral opening 1445 and begin to wrap around the delivery lumen 1440 as the operator advances the elongate portion 1454. Once the anchor portion 1452 is fully deployed, the operator may anchor the anchor portion 1452 against the distal surface of the lumen wall 1470 to assist in the completion of a medical procedure.

Once the desired medical procedure is complete, the operator may then retract the anchor portion back into the delivery lumen 1440 through the lateral opening 1445 by retracting the elongate portion 1454 in a proximal direction relative to the delivery lumen 1440. Thereafter, the operator may remove the delivery lumen 1440 from the opening 1475 in the lumen wall 1470.

In a further embodiment, the distal tip of the anchor member 1450 may be shaped and/or positioned to exit the opening 1445 in the delivery lumen 1440. For example, the distal end of the anchor member 1450 may be curved toward and positioned proximal of the opening 1445 so that as the anchor member 1450 is advanced in a distal direction relative to the delivery lumen 1440, the anchor member 1450 will pass through the opening 1445. In a further embodiment, the tension stored in the anchor portion 1452 may direct the anchor member 1450 out the opening as the anchor member 1450 is advanced in a distal direction relative to the delivery lumen 1440. As the anchor member 1450 passes through the opening, the tension stored in the anchor portion 1452 may be released as the anchor portion 1452 moves from the contracted configuration to an expanded configuration. The user may then use the anchor member 1450 to locate and/or anchor a device relative to the opening by retracting the anchor portion 1452 against a distal surface of the lumen wall as discussed above.

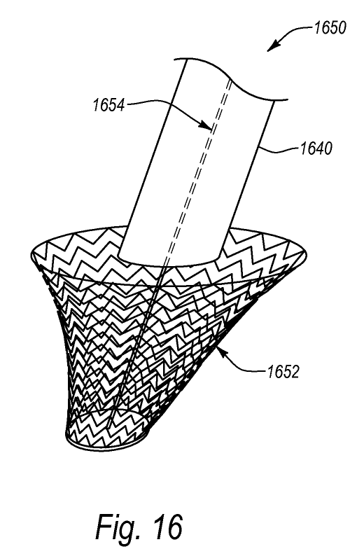

Reference is now made to FIGS. 15A-15B which illustrates an additional example anchor member 1550 in accordance with one embodiment. The anchor member 1550 may be similar in many respects to the anchor members 150, 350, 450, 550, 650, 750, 850, 950, 1050, 1150, 1250, 1350, and 1450 previously described above and shown in FIGS. 1-14 in most respects, wherein certain features will not be described in relation to this configuration wherein those components may function in the manner as described above and are hereby incorporated into this additional configuration described below. Like structures and/or components are given like reference numerals. Additionally, the anchor member 1550 may incorporate at least one component of the anchor members 1650, 1750, 1950, 2050, and 2150 described in connection with FIGS. 16-21, respectively.

FIG. 15A is a cutaway view of the anchor member 1550 disposed within a delivery lumen 1540 in a contracted configuration. FIG. 15B is a cutaway view of the anchor member 1550 with an anchor portion extending at least partially out of the delivery lumen 1540 in an expanded configuration.

In one embodiment, the anchor member 1550 may have an elongate portion 1554 and an anchor portion 1552. For example, the elongate portion 1554 may include a mandrel that extends within the delivery lumen 1540 from a point proximal of the anchor portion 1552, such as near a user, to or just beyond the anchor portion 1552. In a further embodiment, the elongate portion 1554 may be operatively associated with the anchor portion 1552. For example, the elongate portion 1554 may be configured to selectively expand and contract the anchor portion 1552 through a gear-like mechanism.

In one embodiment, the elongate portion 1554 may include a plurality of gear-like teeth 1555 along the length thereof. As used herein, the term “teeth” shall refer to any projection extending from a surface of the elongate portion 1554. In a further embodiment, the teeth 1555 may have a rounded surface and may extend annularly around the elongate portion 1554. The size and shape of the teeth 1555 may be configured to engage corresponding recesses 1553 within projections 1556 of the anchor portion 1552. In addition, anchor member 1550 may have any number of teeth 1555 and corresponding recesses 1553 desired.

A user may operate the elongate portion 1554 to expand and contract the projections 1556 of the anchor portion 1552. For example, in one embodiment, the anchor member 1550 may be configured such that a user may retract the elongate member 1554 in a proximal direction relative to the anchor portion 1552. As a result, the teeth 1555 of the elongate portion 1554 may engage the recesses 1553 of the anchor portion 1552 in order to expand the anchor portion 1552. In addition, the user may contract the anchor portion 1552 by advancing the elongate portion 1554 in a proximal direction relative to the anchor portion 1552.

As mentioned above, the anchor portion 1552 may include a plurality of projections 1556 configured to selectively expand and contract. In one embodiment, the projections 1556 may have a wing-like shape. In a further embodiment, the anchor portion 1552 may have four projections 1556. However, in further embodiments, the anchor portion 1552 may have greater or fewer projections 1556 as desired for particular applications, such as one, two, three, five, six, or more projections 1556.

In a further embodiment, the anchor portion 1552 may be operatively associated with the delivery lumen 1540 to facilitate expansion and contraction of the anchor portion 1552. For example, each projection 1556 of the anchor portion 1552 may be coupled to the delivery lumen 1540 at one or more hinges 1557. In one embodiment, the hinges 1557 may connect to the delivery lumen 1540 and span lateral openings 1545 within the delivery lumen. In a further embodiment, the hinges 1557 may extend through the projections 1556 and the projections 1556 may be configured to rotate about the hinges 1557. As a result, as a user advances and retracts the elongate member 1554 relative to the delivery lumen 1540, the projections 1557 may rotate about the hinges 1557 to expand and contract as explained above.

In one embodiment, the projections 1557 may have an outer surface that is comparable in size and shape to the openings 1545 in the delivery lumen 1540, such that the openings 1545 may be substantially closed when the anchor portion 1552 is in a contracted position. In addition, the projections 1556 may have an upper surface configured to be substantially parallel with a lumen wall when in an expanded position to facilitate anchoring against the lumen wall.

As a result, a user may anchor/located an opening in a body lumen wall by advancing the delivery lumen 1540 and anchor member 1550 through the opening and then expanding the anchor portion 1552 by retracting the elongate portion 1554 relative to the delivery lumen 1540. Thereafter, the user may retract the expanded anchor member 1550 and delivery lumen 1540 in a distal direction to locate/anchor the body lumen opening to facilitate completion of a medical procedure. Once the medical procedure is complete, the user may advance the elongate portion 1554 in a distal direction relative to the delivery lumen 1540 and anchor portion 1552 to retract the projections 1556 back into the delivery lumen 1540. Thereafter, the user may retract the anchor member 1550 and delivery lumen 1540 out of and away from the body lumen opening.

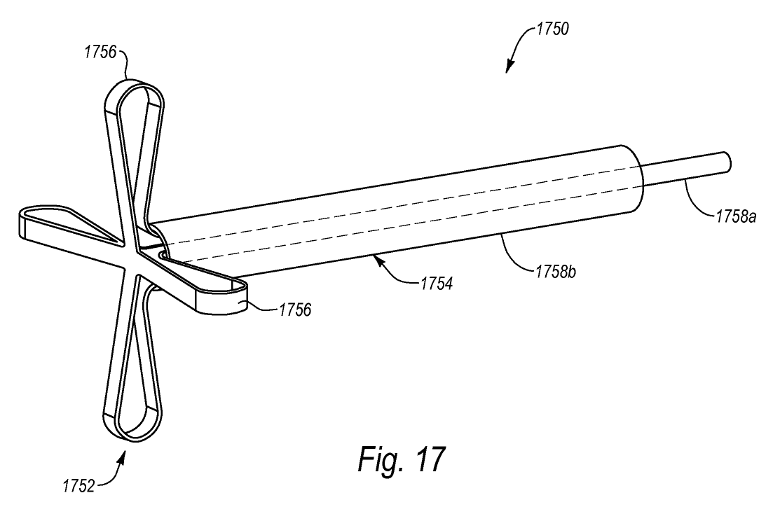

Reference is now made to FIG. 16 which illustrates an additional example anchor member 1650 in accordance with an additional embodiment. The anchor member 1650 may be similar in many respects to the anchor members 150, 350, 450, 550, 650, 750, 850, 950, 1050, 1150, 1250, 1350, 1450, and 1550 previously described above and shown in FIGS. 1-15 in most respects, wherein certain features will not be described in relation to this configuration wherein those components may function in the manner as described above and are hereby incorporated into this additional configuration described below. Like structures and/or components are given like reference numerals. Additionally, the anchor member 1650 may incorporate at least one component of the anchor members 1750, 1950, 2050, and 2150 described in connection with FIGS. 17-21, respectively.

FIG. 16 shows a perspective view of the anchor member 1650 in an expanded configuration. In one embodiment, the anchor member 1650 may include an anchor portion 1652 and an elongate portion 1654. In addition, the anchor member 1650 may be initially disposed within a delivery lumen 1640 in a contracted configuration.

In one embodiment, the anchor portion 1652 of the anchor member 1650 may include a wire mesh basket with a distal end coupled to the elongate portion 1654 and an open proximal end. The anchor portion 1652 may be either self expanding or expandable by some other mechanism. In a further embodiment, the anchor portion 1652 may be initially disposed within the delivery lumen 1640 in a contracted configuration. A user may then advance the delivery lumen 1640 through a body lumen opening. Thereafter, the user may deploy the anchor portion 1652 from a distal opening in the delivery lumen 1640. In one embodiment, the user may deploy the anchor portion 1652 by advancing the elongate portion 1654 in a distal direction relative to the delivery lumen 1640 until the anchor portion 1652 exits the delivery lumen 1640. In a further embodiment, the anchor portion 1652 may expand, either superelastically or by physical force, to its deployed configuration, as shown in FIG. 16. Once the anchor portion 1652 is deployed, the user can retract the elongate portion 1654 in a proximal direction to position the anchor portion 1652 against a distal surface of the body lumen wall to anchor and/or locate the tissue surrounding the body lumen opening to facilitate completion of a medical procedure.

Once the anchor member 1650 is no longer needed, the user may contract the anchor portion 1652 by advancing the delivery lumen 1640 in a distal direction relative to the anchor member 1650 until the anchor portion 1652 re-enters the delivery lumen 1640. In one embodiment, the anchor portion 1652 may fold in a distal direction to facilitate re-entry into the delivery lumen 1640. Once the anchor portion 1652 is retracted into the delivery lumen 1640, the user may retract the delivery lumen 1640 and anchor member 1650 out of and away from the body lumen.

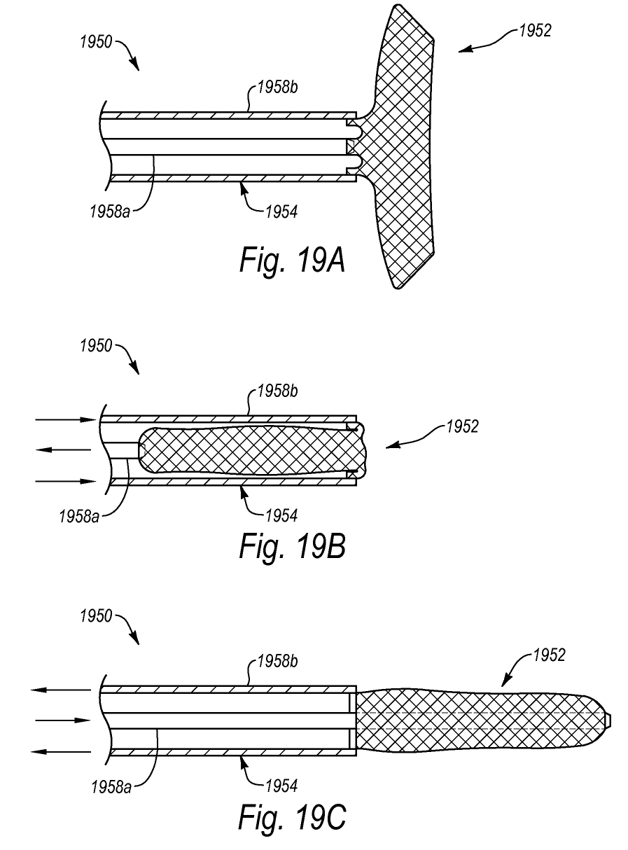

Reference is now made to FIG. 17, which illustrates a perspective view of a yet further example anchor member 1750 in accordance with the present disclosure. The anchor member 1750 may be similar in many respects to the anchor members 150, 350, 450, 550, 650, 750, 850, 950, 1050, 1150, 1250, 1350, 1450, 1550, and 1650 previously described above and shown in FIGS. 1-16 in most respects, wherein certain features will not be described in relation to this configuration wherein those components may function in the manner as described above and are hereby incorporated into this additional configuration described below. Like structures and/or components are given like reference numerals. Additionally, the anchor member 1750 may incorporate at least one component of the anchor members 1950, 2050, and 2150 described in connection with FIGS. 19-21, respectively.

As shown, the anchor member 1750 may have an elongate portion 1754 and an anchor portion 1752. In one embodiment, the elongate portion 1754 may include a plurality of elongate members 1758. For example, the elongate portion 1754 may include a first elongate member 1758a and a second elongate member 1758b. In one embodiment, the first elongate member 1758a may be a mandrel or push/pull wire. In a further embodiment, the first elongate member 1758a may pass at least partially through the second elongate member 1758b. In additional embodiments, the first elongate member 1758a may include one or more shape memory materials, such as nitinol, spring steel, and/or other shape memory alloys. In further embodiments, the first elongate member 1758a may include one or more other metals or polymers.

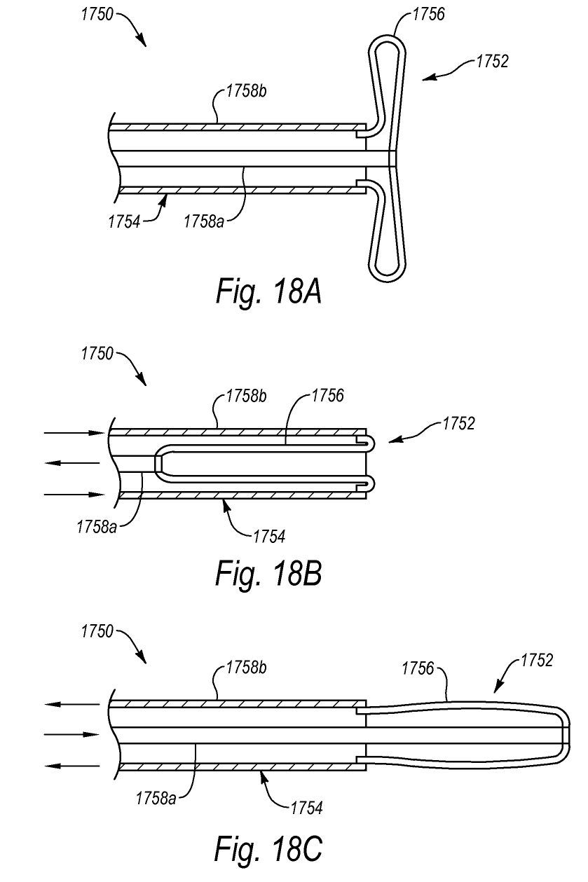

In one embodiment, the second elongate member 1758b may be generally tubular in shape. In a yet further embodiment, the second elongate member 1758b can serve as a guidewire, providing flexibility for easy access and navigation throughout a medical procedure. In an additional embodiment, the second elongate member 1758 may house the anchor portion 1752 within the second elongate member 1758 in an undeployed, contracted configuration until a user desires to deploy the anchor portion 1752 to locate or anchor the tissue surrounding a body lumen opening, as shown in FIG. 18B and described in more detail below. In one embodiment, the second elongate member 1758b may include coiled material to facilitate flexibility. In a further embodiment, the material of the second elongate member 1758b may include stainless steel, nitinol, and/or other shape memory alloys. In yet further embodiments, the materials of the second elongate member 1758b may include any or a combination of a number of other metals or polymers. In additional embodiments, the configuration of the second elongate member 1758b may include a solid tube, a braided wire tube, coiled wire, or other similar structures.

In an additional embodiment, the elongate portion 1754 may be connected to the anchor portion 1752. For example, in one embodiment, the first elongate member 1758a may be connected to a distal end of the anchor portion 1752 and the second elongate member 1758b may be connected to a proximal end of the anchor portion 1752. The connection between the anchor portion 1752 and elongate portion 1754 can be achieved through welding, adhering, or any other fastening mechanism. In further embodiments, the anchor portion 1752 and elongate portion 1754 can be integrally formed together. As a result, the elongate portion 1754 can be used to deploy and undeploy the anchor portion 1752 as desired by a user.

The anchor portion 1752 may be configured to locate or anchor against the distal surface of tissue surrounding a body lumen opening. In one embodiment, the anchor portion 1752 may have one or more projections 1756 extending away from the longitudinal axis of the elongate portion 1754. In a further embodiment, the projections 1756 may have a deployed, expanded configuration, as shown in FIG. 17, and one or more undeployed, contracted configurations, as shown in FIGS. 18B and 18C, which will be discussed in greater detail below. In one embodiment, each projection 1756 may have a wire or ribbon-like or loop-like shape. In further embodiments, each projection 1756 may have any shape or size desired for a particular application. In addition, the anchor portion 1752 may have any number of projections 1756 desired. For example, although FIG. 17 illustrates the anchor portion 1752 having four projections 1756, in further embodiments, the anchor portion 1752 may have any number of projections 1756, such as one, two, three, five, six, or more projections 1756.

In one embodiment, the projections 1756 may include one or more shape memory materials and may be heat set to have a memory shape. For example, the projections 1756 may be heat set in their expanded configuration shown in FIG. 17. As a result, when the anchor portion 1752 is deployed, it may superelastically move to its expanded configuration. Thereafter, a user may apply a force to the anchor portion 1752 to deform the projections 1756 away from their memory shape and contract the anchor portion 1752. In a yet further embodiment, the projections 1756 may have a contracted memory shape and the user may apply a force to the anchor portion 1752 to move the anchor portion 1752 to an expanded configuration.

Reference is now made to FIGS. 18A-18C, which illustrate additional views of the anchor member 1750 of FIG. 17 in various configurations. In particular, FIG. 18A illustrates a side view of the anchor member 1750 with the anchor portion 1752 in an expanded configuration, FIG. 18B illustrates a side view of the anchor member 1750 in a first contracted configuration, and FIG. 18C illustrates a side view of the anchor member 1750 in a second contracted configuration.

As shown in FIG. 18A, the anchor portion 1752 of the anchor member 1750 can have an expanded configuration in which the projections extend substantially perpendicularly away from the longitudinal axis of the elongate member 1754. In one embodiment, the expanded configuration of the anchor portion 1752 may be formed when the elongate projections 1756 fold roughly upon themselves with the bend of each projection 1756 extending radially outwardly.

As shown in FIG. 18B and 18C, the anchor portion 1752 may have a variety of contracted configurations. For example, as shown in FIG. 18B, the anchor portion 1752 may have a first contracted configuration in which the projections 1756 are retracted into the second elongate member 1758b by moving the first elongate member 1758a in a proximal direction relative to the second elongate member 1758b, or moving the second elongate member 1758b in a distal direction relative to the first elongate member 1758a, or any combination thereof. The first contracted configuration shown in FIG. 18B may facilitate the delivery of the anchor member 1750 into or retraction of the anchor member 1750 from a body lumen opening. For example, a user may maintain tension in the first elongate member 1758a as she advances the anchor member 1750 at least partially through a body lumen opening. Thereafter, in one embodiment, the user may release the tension on the first elongate member 1758a, after which the anchor portion may move superelastically to the expanded configuration shown in FIG. 18A. In a yet further embodiment, the user may facilitate deployment of the anchor portion 1752 by advancing the first elongate member 1758a distally with respect to the second elongate member 1758b.

As shown in FIG. 18C, the anchor portion 1752 may have a second contracted configuration in which the projections 1756 are elongated and drawn radially inwardly by moving the first elongate member 1758a in a distal direction relative to the second elongate member 1758b, or moving the second elongate member 1758b in a proximal direction relative to the first elongate member 1758a, or any combination thereof. The second contracted configuration shown in FIG. 18C may facilitate the delivery of the anchor member 1750 into or retraction of the anchor member 1750 from a body lumen opening. For example, a user may maintain the distal force in the first elongate member 1758a as she advances the anchor member 1750 at least partially through a body lumen opening. Thereafter, the user may release the force on the first elongate member 1758a, after which the anchor portion 1752 may move superelastically to the expanded configuration shown in FIG. 18A.

Once a medical procedure is complete, the user may return the anchor portion 1752 to its first contracted configuration shown in FIG. 18B or to its second contracted configuration shown in FIG. 18C, as described above, thereby minimizing contact between the anchor portion 1752 and an external sheath or a tissue track as the anchor member 1750 is withdrawn out of and away from the body lumen.

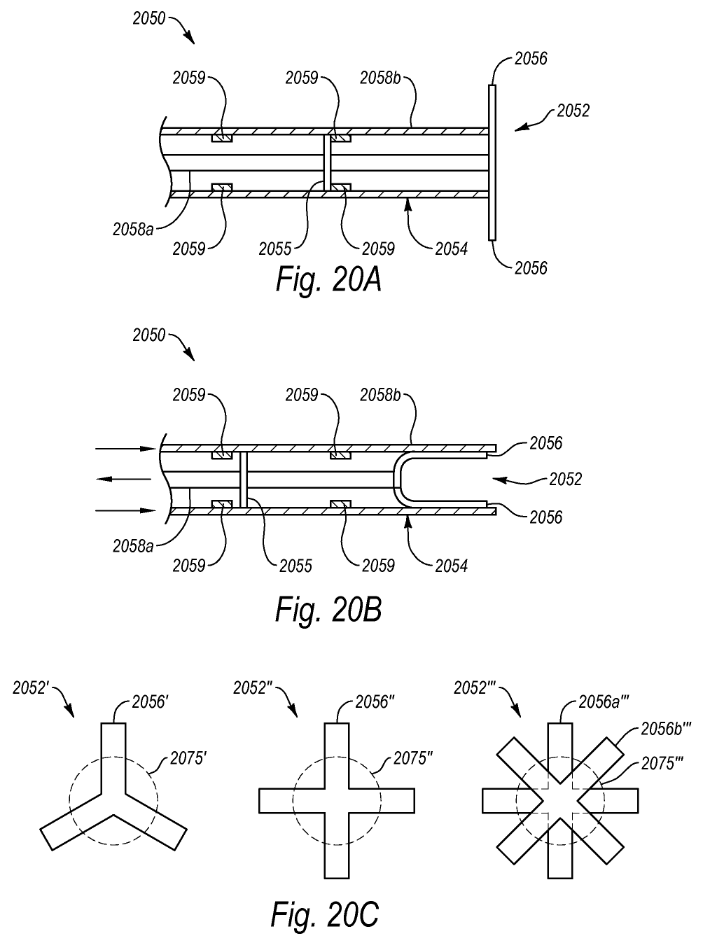

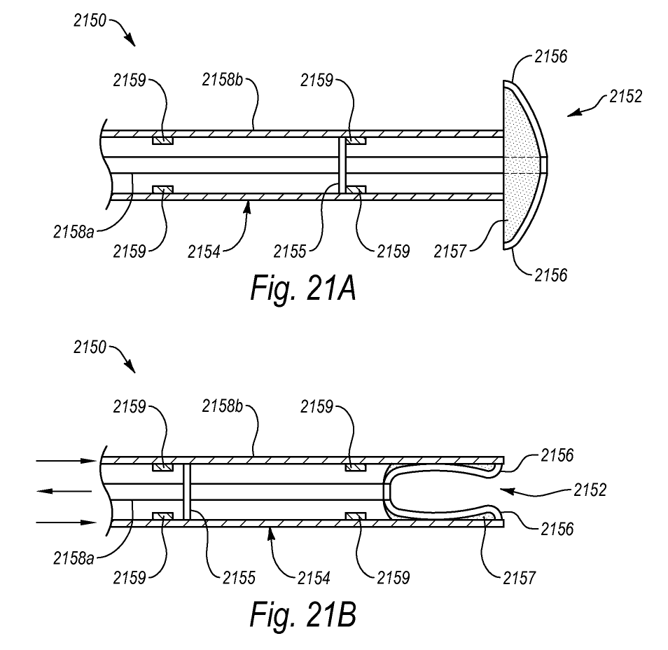

Reference is now made to FIGS. 19A-19C, which illustrate a yet further example anchor member 1950 in accordance with the present disclosure. The anchor member 1950 may be similar in many respects to the anchor members 150, 350, 450, 550, 650, 750, 850, 950, 1050, 1150, 1250, 1350, 1450, 1550, 1650, and 1750 previously described above and shown in FIGS. 1-18 in most respects, wherein certain features will not be described in relation to this configuration wherein those components may function in the manner as described above and are hereby incorporated into this additional configuration described below. Like structures and/or components are given like reference numerals. Additionally, the anchor member 1950 may incorporate at least one component of the anchor members 2050, and 2150 described in connection with FIGS. 20-21, respectively.

As shown, the anchor member 1950 may have an elongate portion 1954 and an anchor portion 1952. In one embodiment, the elongate portion 1954 may include a plurality of elongate members 1958. For example, the elongate portion 1954 may include a first elongate member 1958a and a second elongate member 1958b. In one embodiment, the first elongate member 1958a may be a mandrel or push/pull wire. In a further embodiment, the first elongate member 1958a may pass at least partially through the second elongate member 1958b.

In one embodiment, the second elongate member 1958b may be generally tubular in shape. In a yet further embodiment, the second elongate member 1958b can serve as a guidewire, providing flexibility for easy access and navigation throughout a medical procedure. In an additional embodiment, the second elongate member 1958 may house the anchor portion 1952 within the second elongate member 1958 in an undeployed, contracted configuration until a user desires to deploy the anchor portion 1952 to locate or anchor the tissue surrounding a body lumen opening as shown in FIG. 19B and described in more detail below.

In an additional embodiment, the elongate portion 1954 may be connected to the anchor portion 1952. For example, in one embodiment, the first elongate member 1958a may be connected to a distal end of the anchor portion 1952 and the second elongate member 1958b may be connected to a proximal end of the anchor portion 1952. The connection between the anchor portion 1952 and elongate portion 1954 can be achieved through welding, adhering, or any other fastening mechanism. As a result, the elongate portion 1954 can be used to deploy and undeploy the anchor portion 1952 as desired by a user.

The anchor portion 1952 may be configured to locate or anchor against the tissue surrounding a body lumen opening. In one embodiment, the anchor portion 1952 may include a mesh structure, such as a braided wire mesh. The anchor portion 1952 may have a deployed, expanded configuration, as shown in FIG. 19A, and one or more undeployed, contracted configurations, as shown in FIGS. 19B and 19C, which will be discussed in greater detail below. In one embodiment, the mesh structure of the anchor portion 1952 may have a basket-like shape with an open end connected to the second elongate member 1958b and the closed end connected to the first elongate member 1958a. In further embodiments, the mesh structure of the anchor portion 1952 can have any shape or size desired for a particular application.

In one embodiment, the anchor portion 1952 may include one or more shape memory materials and may be heat set to have a memory shape. For example, the anchor portion 1952 may be heat set in its expanded configuration shown in FIG. 19A. As a result, when the anchor portion 1952 is deployed, it may superelastically move to its expanded configuration. Thereafter, a user may apply a force to the anchor portion 1952 to deform the mesh structure away from its memory shape and contract the anchor portion 1952. In a yet further embodiment, the anchor portion 1952 may have a contracted memory shape and the user may apply a force to the anchor portion 1952 to move the anchor portion 1952 to an expanded configuration.

FIGS. 19A-19C illustrate views of the anchor member 1950 in various expanded and contracted configurations. In particular, FIG. 19A illustrates a side view of the anchor member 1950 with the anchor portion 1952 in an expanded configuration, FIG. 19B illustrates a side view of the anchor member 1950 in a first contracted configuration, and FIG. 19C illustrates a side view of the anchor member 1950 in a second contracted configuration.

As shown in FIG. 19A, the anchor portion 1952 of the anchor member 1950 can have an expanded configuration in which the mesh structure extends substantially perpendicularly away from the longitudinal axis of the elongate portion 1954. In one embodiment, the expanded configuration of the anchor portion 1952 may be formed when the mesh structure forms a ring-like, disc-like, or donut-like shape with portions thereof extending radially outwardly.

As mentioned, the anchor portion 1952 may have a variety of contracted configurations. For example, as shown in FIG. 19B, the anchor portion 1952 may have a first contracted configuration in which the anchor portion 1952 is retracted into the second elongate member 1958b by moving the first elongate member 1958a in a proximal direction relative to the second elongate member 1958b, or moving the second elongate member 1958b in a distal direction relative to the first elongate member 1958a, or any combination thereof. The first contracted configuration shown in FIG. 19B may facilitate the delivery of the anchor member 1950 into or retraction of the anchor member 1950 from a body lumen opening. For example, a user may maintain tension in the first elongate member 1958a as she advances the anchor member 1950 at least partially through a body lumen opening. Thereafter, in one embodiment, the user may release the tension on the first elongate member 1958a, after which the anchor portion may move superelastically to the expanded configuration shown in FIG. 19A. In a yet further embodiment, the user may facilitate deployment of the anchor portion 1952 by advancing the first elongate member 1958a distally with respect to the second elongate member 1958b.

As shown in FIG. 19C, the anchor portion 1952 may have a second contracted configuration in which the anchor portion 1952 is elongated and drawn radially inwardly by moving the first elongate member 1958a in a distal direction relative to the second elongate member 1958b, or moving the second elongate member 1958b in a proximal direction relative to the first elongate member 1958a, or any combination thereof. The second contracted configuration shown in FIG. 19C may facilitate the delivery of the anchor member 1950 into or retraction of the anchor member 1950 from a body lumen opening. For example, a user may maintain the distal force in the first elongate member 1958a as she advances the anchor member 1950 at least partially through a body lumen opening. Thereafter, the user may release the force on the first elongate member 1958a, after which the anchor portion 1952 may move superelastically to the expanded configuration shown in FIG. 19A.

Once expanded, the anchor portion 1952 may be used to anchor against a distal surface of a body lumen wall near a body lumen opening and to help position the distal end of another medical device, such as a vessel closure system, to the external surface of the body lumen wall. As a result, the anchor portion 1952 can assist in completing a medical procedure. Once a medical procedure is complete, the user may return the anchor portion 1952 to its first contracted configuration shown in FIG. 19B or to its second contracted configuration shown in FIG. 19C, as described above, and withdraw the anchor member 1950 out of and away from the body lumen.

Reference is now made to FIGS. 20A-20C, which illustrate a yet further example anchor member 2050 in accordance with the present disclosure. The anchor member 2050 may be similar in many respects to the anchor members 150, 350, 450, 550, 650, 750, 850, 950, 1050, 1150, 1250, 1350, 1450, 1550, 1650, 1750, and 1950 previously described above and shown in FIGS. 1-19 in most respects, wherein certain features will not be described in relation to this configuration wherein those components may function in the manner as described above and are hereby incorporated into this additional configuration described below. Like structures and/or components are given like reference numerals. Additionally, the anchor member 2050 may incorporate at least one component of the anchor member 2150 described in connection with FIG. 21, respectively.

As shown, the anchor member 2050 may have an elongate portion 2054 and an anchor portion 2052. In one embodiment, the elongate portion 2054 may include a plurality of elongate members 2058. For example, the elongate portion 2054 may include a first elongate member 2058a and a second elongate member 2058b. In one embodiment, the first elongate member 2058a may be a mandrel or push/pull wire. In a further embodiment, the first elongate member 2058a may pass at least partially through the second elongate member 2058b.

In a yet further embodiment, the first elongate member 2058a may have a collar 2055 disposed at a particular position along the length of the first elongate member 2058a. In one embodiment, the collar 2055 may be configured to provide limitations on the relative movement between the first elongate member 2058a and second elongate member 2058b, as will be discussed in greater detail below. The collar 2055 may have any size and shape desired to interact with corresponding features of the second elongate member 2058b. In one embodiment, the collar 2055 may have a disc-like shape with a rigid or semi-rigid configuration.

In one embodiment, the second elongate member 2058b may be generally tubular in shape. In a yet further embodiment, the second elongate member 2058b can serve as a guidewire, providing flexibility for easy access and navigation throughout a medical procedure. In an additional embodiment, the second elongate member 2058 may house the anchor portion 2052 within the second elongate member 2058 in an undeployed, contracted configuration until a user desires to deploy the anchor portion 2052 to locate or anchor the tissue surrounding a body lumen opening as shown in FIG. 20B and described in more detail below

In a further embodiment, the second elongate member 2058b may have one or more stops 2059 disposed along the inner surface thereof. The stops 2059 may be positioned to limit movement of the first elongate member 2058a by preventing the collar 2055 of the first elongate member 2058a from passing the stops 2059. The stops 2059 may have any shape or size necessary to prevent movement by the collar 2055. For example, the stops 2059 may include any feature extending from the inner surface of the second elongate member 2058b. In one embodiment, the stops may have a generally rectangular cross-section and may extend annularly around the inner surface of the second elongate member 2058b. In further embodiments, the stops 2059 may have any cross-sectional shape or size desired. In yet further embodiments, the stops 2059 may not extend annularly around the inner surface of the second elongate member 2058b, but may extend only a portion of the way around the inner surface of the second elongate member 2058b.

In addition, the second elongate member 2058b may have one or more proximal stops 2059 positioned to limit proximal movement by the collar 2055 and one or more distal stops 2059 positioned to limit distal movement by the collar 2055. As a result, the stops 2059 can confine the movement of the first elongate member 2058a and thereby confine the movement of the anchor portion 2052. In one embodiment, the stops 2059 may allow sufficient distal movement of the first elongate member 3058a and anchor portion 2052 to allow the anchor portion 2052 to fully deploy. In a further embodiment, the stops 2059 may allow sufficient proximal movement of the first elongate member 2058a and anchor portion 2052 to retract fully into the second elongate member 2058b.

In an additional embodiment, the elongate portion 2054 may be connected to the anchor portion 2052. For example, in one embodiment, the first elongate member 2058a may be connected near the center of the anchor portion 2052. In further embodiments, and as shown in FIG. 20A, the second elongate member 2058b may not be connected to the anchor portion 2052 in order to facilitate relative movement between the second elongate member 2058b and the anchor portion 2052. As a result, the first elongate member 2058a can be used to deploy and/or undeploy the anchor portion 2052 as desired by a user.

The anchor portion 2052 may be configured to locate or anchor against the tissue surrounding a body lumen opening. In one embodiment, the anchor portion 2052 may have one or more projections 2056 extending away from the longitudinal axis of the elongate portion 2054. In a further embodiment, the projections 2056 may have one or more deployed, expanded configurations, as shown in FIGS. 20A and 20C, and an undeployed, contracted configuration, as shown in FIG. 20B, which will be discussed in greater detail below.

In one embodiment, each projection 2056 may have a wire, strip-like, or ribbon-like shape with a fixed end connected to the first elongate member 2058a and a free end configured to extend radially outwardly to assist in anchoring or locating tissue surrounding a body lumen opening. For example, the projections 2056 of the anchor portion 2052 may be formed by one or more strips of material. In an additional embodiment, the anchor portion 2052 may be cut from a sheet of material. The sheet of material may be cut into any of a variety of configurations to have multiple projections 2056 and designs, as shown, for example, in FIG. 20C. In further embodiments, each projection 2056 may have any shape, size, or configuration desired for a particular application. In addition, the anchor portion 2052 may have any number of projections 2056 desired. For example, although FIG. 20 illustrates the anchor portion 2052 having two projections 2056, in further embodiments, the anchor portion 2052 may have any number of projections 2056, such as one, three, four, five, six, or more projections 2056.

In one embodiment, the projections 2056 may include one or more shape memory materials, such as spring steel, nitinol, and/or other shape memory alloys, and may be heat set to have a memory shape. For example, the projections 2056 may be heat set in their expanded configuration shown in FIG. 20A. As a result, when the anchor portion 2052 is deployed, it may superelastically move to its expanded configuration. Thereafter, a user may apply a force to the anchor portion 2052 to deform the projections 2056 away from their memory shape and move the anchor portion 2052 into a contracted configuration, as shown in FIG. 20B. In a yet further embodiment, the projections 2056 may have a contracted memory shape and the user may apply a force to the anchor portion 2052 to move the anchor portion 2052 to an expanded configuration.

As mentioned, FIGS. 20A-20B illustrate views of the anchor member 2050 in various configurations. In particular, FIG. 20A illustrates a side view of the anchor member 2050 with the anchor portion 2052 in an expanded configuration and FIG. 20B illustrates a side view of the anchor member 2050 in a contracted configuration. As shown in FIG. 20A, the anchor portion 2052 of the anchor member 2050 can have an expanded configuration in which the projections extend substantially perpendicularly away from the longitudinal axis of the elongate member 2054 in a radially outward direction.

As shown in FIG. 20B, the anchor portion 2052 may have a contracted configuration in which the projections 2056 are retracted into the second elongate member 2058b by moving the first elongate member 2058a in a proximal direction relative to the second elongate member 2058b, or moving the second elongate member 2058b in a distal direction relative to the first elongate member 2058a, or any combination thereof. The contracted configuration shown in FIG. 20B may facilitate the delivery of the anchor member 2050 into or retraction of the anchor member 2050 from a body lumen opening. For example, a user may maintain tension in the first elongate member 2058a as she advances the anchor member 2050 at least partially through a body lumen opening. Thereafter, in one embodiment, the user may release the tension on the first elongate member 2058a or advance the first elongate member 2058a in a distal direction relative to the second elongate member 2058b, after which the anchor portion may move superelastically to the expanded configuration shown in FIG. 20A.

Once a medical procedure is complete, the user may return the anchor portion 2052 to its first contracted configuration shown in FIG. 20B and withdraw the anchor member 2050 out of and away from the body lumen.

As shown in FIG. 20C, the anchor portion 2052 may have a plurality of different expanded configurations. FIG. 20C illustrates a number of example anchor portions 2052′, 2052″, 2052″ in accordance with the present disclosure. In one embodiment, the anchor member 2050 may have an anchor portion 2052′ with three projections 2056′. In a further embodiment, the projections 2056′ may be spaced evenly about the anchor portion 2052′. In an additional embodiment, the anchor member 2050 may have an anchor portion 2052″ with four projections 2056″. In a further embodiment, the projections 2056″ may form a shape similar to an ‘x’. In a yet further embodiment, the anchor member 2050 may have an anchor portion 2052′″ with multiple layers of projections 2056′″. For example, the anchor portion 2052′″ may include a first set of projections 2056a′″ and a second set of projections 2056′″. Each set may include any number of projections 2056′″ desired for a particular application. In addition, the separate sets of projections 2056′″ may overlap directly, may be cut from the same piece of material, or may be longitudinally separated by any distance desired. A user may use the anchor portions 2052′, 2052″, 2052′″ to anchor or locate tissue surrounding the body lumen openings 2075′, 2075″, 2075′″.

Reference is now made to FIGS. 21A-21B, which illustrate a yet further example anchor member 2150 in accordance with the present disclosure. The anchor member 2150 may be similar in many respects to the anchor members 150, 350, 450, 550, 650, 750, 850, 950, 1050, 1150, 1250, 1350, 1450, 1550, 1650, 1750, 1950, and 2050 previously described above and shown in FIGS. 1-20 in most respects, wherein certain features will not be described in relation to this configuration wherein those components may function in the manner as described above and are hereby incorporated into this additional configuration described below. Like structures and/or components are given like reference numerals.

As shown, the anchor member 2150 may have an elongate portion 2154 and an anchor portion 2152. In one embodiment, the elongate portion 2154 may include a plurality of elongate members 2158. For example, the elongate portion 2154 may include a first elongate member 2158a and a second elongate member 2158b. In one embodiment, the first elongate member 2158a may be a mandrel or push/pull wire. In a further embodiment, the first elongate member 2158a may pass at least partially through the second elongate member 2158b.

In a yet further embodiment, the first elongate member 2158a may have a collar 2155 disposed at a particular position along the length of the first elongate member 2158a. In one embodiment, the collar 2155 may be configured to provide limitations on the relative movement between the first elongate member 2158a and second elongate member 2158b to prevent excessive movement in either the distal or proximal direction relative to the second elongate member 2158b.

In one embodiment, the second elongate member 2158b may be generally tubular in shape. In a further embodiment, the second elongate member 2158b may have one or more stops 2159 disposed along the inner surface thereof. The stops 2159 may be positioned and configured to limit movement of the first elongate member 2158a. In one embodiment, the stops 2159 may allow sufficient distal movement of the first elongate member 2058a and anchor portion 2152 to allow the anchor portion 2152 to fully deploy and sufficient proximal movement of the first elongate member 2158a and anchor portion 2152 to retract fully into the second elongate member 2158b.

In an additional embodiment, the elongate portion 2154 may be connected to the anchor portion 2152. For example, in one embodiment, the first elongate member 2158a may be connected near the center of the anchor portion 2152. In further embodiments, and as shown in FIG. 21 A, the second elongate member 2158b may not be connected to the anchor portion 2152 in order to facilitate relative movement between the second elongate member 2158b and the anchor portion 2152.

The anchor portion 2152 may be configured to locate or anchor against the tissue surrounding a body lumen opening. In one embodiment, the anchor portion 2152 may have one or more projections 2156 extending away from the longitudinal axis of the elongate portion 2154. The projections 2156 may have a deployed, expanded configuration, as shown in FIG. 21A and an undeployed, contracted configuration, as shown in FIG. 21B.

In one embodiment, each projection 2156 may have a wire or ribbon-like shape with a fixed end connected to the first elongate member 1758a and a free end configured to extend radially outwardly to assist in anchoring or locating tissue surrounding a body lumen opening. In a further embodiment, the deployed configuration of each projection 2156 may have a slightly curved shape in which the projection 2156 curves back in a proximal direction, as shown in FIG. 21A. In addition, the anchor portion 2152 may have any number of projections 2156 desired. For example, although FIG. 21 illustrates the anchor portion 2152 having two projections 2156, in further embodiments, the anchor portion 2152 may have any number of projections 2156, such as one, three, four, five, six, or more projections 2156.

In yet further embodiments, the anchor portion 2152 may include a membrane 2157 disposed on the one or more projections 21560 of the anchor portion 2152. In one embodiment, the membrane 2157 may be configured to provide temporary hemostasis when the anchor portion 2152 is deployed within a body lumen. In yet further embodiments, the membrane 2157 may be at least partially impermeable. In one embodiment, the membrane 2157 may include one or more polymers or fabrics configured to be at least partially expandable. As a result, the anchor portion 2152 may have an umbrella-like configuration.

In one embodiment, the projections 2156 may include one or more shape memory materials and may be heat set to have a memory shape. For example, the projections 2156 may be heat set in their expanded configuration shown in FIG. 21A. As a result, when the anchor portion 2152 is deployed, it may superelastically move to its expanded configuration. Thereafter, a user may apply a force to the anchor portion 2152 to deform the projections 2156 away from their memory shape and move the anchor portion 2152 into a contracted configuration, as shown in FIG. 21B. In a yet further embodiment, the projections 2156 may have a contracted memory shape and the user may apply a force to the anchor portion 2152 to move the anchor portion 2152 to an expanded configuration.

As shown in FIG. 21B, the anchor portion 2152 may have a contracted configuration in which the projections 2156 are retracted into the second elongate member 2158b by moving the first elongate member 2158a in a proximal direction relative to the second elongate member 2158b, or moving the second elongate member 2158b in a distal direction relative to the first elongate member 2158a, or any combination thereof. The contracted configuration shown in FIG. 2IB may facilitate the delivery of the anchor member 2150 into or retraction of the anchor member 2150 from a body lumen opening. For example, a user may advance the anchor member 2150 in its contracted configuration at least partially through a body lumen opening. Thereafter, in one embodiment, the user may release the tension on the first elongate member 2158a or advance the first elongate member 2158a in a distal direction relative to the second elongate member 2158b, after which the anchor portion 2152 may move superelastically to the expanded configuration shown in FIG. 21A.

Once a medical procedure is complete, the user may return the anchor portion 2152 to its contracted configuration shown in FIG. 2IB by retracting the anchor portion 2152 into the second elongate member 2158b, as described above, and withdraw the anchor member 2150 out of and away from the body lumen.

The present disclosure may be embodied in other specific forms without departing from its spirit or essential characteristics. The described embodiments are to be considered in all respects only as illustrative and not restrictive. The scope of the disclosure is, therefore, indicated by the appended claims rather than by the foregoing description. All changes which come within the meaning and range of equivalency of the claims are to be embraced within their scope.

What is claimed is:

1. An anchor member configured to locate and/or anchor tissue surrounding a body lumen opening comprising:
an elongate portion configured to be manipulated by a user; and
an anchor portion having one or more contracted configurations capable of passing through a body lumen opening and having one or more expanded configurations capable of anchoring tissue surrounding a body lumen opening.

2. The anchor member of claim 1, wherein the anchor member includes one or more shape memory materials.

3. The anchor member of claim 1, wherein the anchor member comprises a shape memory wire, and wherein the anchor portion comprises a portion of the shape memory wire being heat set into an aneurysm coil or bird's nest having a plurality of non-uniformly shaped and oriented loops of the shape memory wire.

4. The anchor member of claim 1, wherein the anchor member comprises a shape memory wire, and wherein the anchor portion comprises a portion of the shape memory wire being heat set into a spiraling configuration having a plurality of spirals extending radially outwardly from a longitudinal axis of the elongate portion.

5. The anchor member of claim 4, further comprising a delivery lumen with a lateral opening configured to allow deployment of the anchor portion therethrough.

6. The anchor member of claim 1, wherein the anchor portion comprises an expandable mesh structure.

7. The anchor member of claim 1, wherein the anchor portion comprises a plurality of hinged struts configured to expand radially outwardly, and wherein the elongate portion comprises a first elongate member disposed through a generally tubular second elongate member.

8. The anchor member of claim in 7, wherein the first elongate member is connected to a distal end of the anchor portion and the second elongate member is connected to a proximal end of the anchor portion and wherein selective relative movement between the first elongate member and second elongate member causes the anchor portion to selectively expand and contract.

9. The anchor member of claim 8, further comprising a flexible membrane disposed around at least a portion of the anchor portion and configured to expand with the hinged struts.

10. The anchor member of claim 1, wherein the anchor portion comprises an expandable coil of shape memory wire.

11. The anchor member of claim 1, wherein the anchor member is generally tubular having a straight portion forming the elongate portion and a looped portion forming the anchor portion configured to pass through an opening by rotation of the anchor member.

12. The anchor member of claim 11, wherein the anchor member is initially disposed in a delivery lumen in a contracted configuration and is configured to superelastically deploy through a lateral opening in the delivery lumen into an expanded configuration.

13. The anchor member of claim 1, wherein the elongate portion comprises a mandrel having one or more teeth disposed along a length thereof and the anchor portion comprises one or more rotatable projections each having one or more recesses with sizes and shapes corresponding to the sizes and shapes of the one or more teeth of the elongate portion.

14. The anchor member of claim 13, wherein the elongate portion is configured to rotate the one or more projections of the anchor portion by moving longitudinally relative to the anchor portion with the teeth of the elongate portion engaging the recesses of the one or more projections.

15. The anchor member of claim 14, further comprising a delivery lumen and one or more hinges connected to the delivery lumen and passing through the one or more projections to facilitate rotation of the projections about the hinges.

16. The anchor member of claim 15, wherein the projections have a wing-like shape.

17. The anchor member of claim 1, wherein the anchor portion comprises a wire mesh basket having an open proximal end and a closed distal end.

18. The anchor member of claim 17, wherein a distal end of the elongate portion is connected to the distal end of the anchor portion.

19. The anchor member of claim 1, wherein the elongate portion comprises a first elongate member and a generally tubular second elongate member, and wherein the first elongate member is disposed at least partially through the second elongate member.

20. The anchor member of claim 19, wherein the anchor portion comprises one or more projections configured to be movable between a contracted configuration and an expanded configuration.

21. The anchor member of claim 19, wherein the distal end of the first elongate member is connected to a distal end of the anchor portion and a distal end of the second elongate member is connected to a proximal end of the anchor portion.

22. The anchor member of claim 19, wherein a distal end of the first elongate member is connected to a center or proximal end of the anchor portion, and wherein the anchor portion has a free outside or distal end.

23. The anchor member of claim 20, wherein the one or more projections have a ribbon- or strip-like configuration with one end thereof connected to the first elongate member and an opposite end thereof connected to the second elongate member.

24. The anchor member of claim 21, wherein the anchor portion comprises a wire mesh structure.

25. The anchor member of claim 19, wherein the anchor portion has a first contracted configuration wherein the anchor portion is retracted into the second elongate member and a second contracted configuration wherein the anchor portion is elongated in a distal direction.

26. The anchor member of claim 19, wherein the first elongate member further comprises a collar positioned thereon and the second elongate member further comprises one or more stops disposed on the inner surface thereof, and wherein the collar and stops are configured to limit relative longitudinal movement between the first elongate member and the second elongate member.

27. The anchor member of claim 26, wherein the collar and stops are configured to allow sufficient distal movement of the first elongate member to deploy the anchor portion and sufficient proximal movement of the first elongate member to retract the anchor portion into the second elongate member.

28. The anchor member of claim 22, further comprising a membrane disposed on at least a portion of the anchor portion.

29. The anchor member of claim 28, wherein the one or more projections have an expanded configuration that at least partially curves in a proximal direction.

30. The anchor member of claim 19, wherein the second elongate member comprises a guidewire.

31. The anchor member of claim 20, comprising a number of projections chosen from the group consisting of two, three, and four.

32. A closure system comprising:
a handle member;
a tube set configured to deliver and/or deploy a closure element, the tube set having a distal end and a proximal end, the proximal end of the tube set being coupled to the handle member;
a plunger member movably coupled to the handle member; and
an anchor member disposed at least partially within the tube set comprising:
an elongate portion configured to be manipulated by a user; and
an anchor portion having one or more contracted configurations capable of passing through a body lumen opening and having one or more expanded configurations capable of anchoring tissue surrounding a body lumen opening.
